SEQUENCE LISTING (43 sequences)

SEQ ID NO: 1 - DNA, 21 bp, Artificial
  gRNA S1 sequnce of Vlk_PCS_S
aagcgaaatcaacagccggag

SEQ ID NO: 2 - DNA, 19 bp, Artificial
  gRNA S2 sequnce of Vlk_PCS_S
ggcattcaagaccatggaa

SEQ ID NO: 3 - DNA, 19 bp, Artificial
  gRNA T1 sequnce of Vlk_PCS_T
cgagttcttccgtcgcctc

SEQ ID NO: 4 - DNA, 19 bp, Artificial
  gRNA T2 sequnce of Vlk_PCS_T
ggcattcaagaccatagaa

SEQ ID NO: 5 - DNA, 19 bp, Artificial
  gRNA_ST1 sequnce of Vlk_PCS_ST
tagaagcgaaatcaacagc

SEQ ID NO: 6 - DNA, 19 bp, Artificial
  gRNA_ST2 sequnce of Vlk_PCS_ST
gccatccagaatggaacaa

SEQ ID NO: 7 - DNA, 28 bp, Artificial
  F_HMA alpha primer sequence
gaaacaaagaagttgagcaagagctatt

SEQ ID NO: 8 - DNA, 28 bp, Artificial
  R_HMA alpha primer sequence
agccttagtgagatgatttataacacaa

SEQ ID NO: 9 - DNA, 28 bp, Artificial
  F_HMA beta primer sequence
gacacaaagaatctgagcaagagctatt

SEQ ID NO: 10 - DNA, 28 bp, Artificial
  R_HMA beta primer sequence
agctagagtaggaccacacattaattct

SEQ ID NO: 11 - DNA, 23 bp, Artificial
  F_PCSs primer sequence
aaatggcgatggcgggtttgtat

SEQ ID NO: 12 - DNA, 28 bp, Artificial
  R_PCSs primer sequence
gtcgggaaggattagaacacaaattcac

SEQ ID NO: 13 - DNA, 23 bp, Artificial
  F_PCSt primer sequence
aatggcgatggcgggtttatatc

SEQ ID NO: 14 - DNA, 26 bp, Artificial
  R_PCSt primer sequence
agtccggaaggataggaacacagatt

SEQ ID NO: 15 - DNA, 20 bp, Artificial
  gRNA_A1 sequnce of Vlk_HMA_A6
tctttcttaccaatttgttg

SEQ ID NO: 16 - DNA, 20 bp, Artificial
  gRNA_A2 sequnce of Vlk_HMA_A6
tgtttgtacaagcttttaga

SEQ ID NO: 17 - DNA, 21 bp, Artificial
  gRNA_A3 sequnce of Vlk_HMA_A6
atggtaacttcaataattata

SEQ ID NO: 18 - DNA, 19 bp, Artificial
  gRNA_A4 of Vlk_HMA_A6
aagcaagcataagagtgaa

SEQ ID NO: 19 - DNA, 19 bp, Artificial
  gRNA_A5 sequnce of Vlk_HMA_A6
ccacacctctaaaaataat

SEQ ID NO: 20 - DNA, 19 bp, Artificial
  gRNA_A6 sequnce of Vlk_HMA_A6
tcatataaattgggacaaa

SEQ ID NO: 21 - DNA, 20 bp, Artificial
  gRNA_B1 sequence of Vlk_HMA_B6
caatttgttgctgagaaatg

SEQ ID NO: 22 - DNA, 20 bp, Artificial
  gRNA_B2 sequence of Vlk_HMA_B6
agtggagaaaagatgaagaa

SEQ ID NO: 23 - DNA, 21 bp, Artificial
  gRNA_B3 sequence of Vlk_HMA_B6
atggtaactacaataattata

SEQ ID NO: 24 - DNA, 19 bp, Artificial
  gRNA_B4 sequence of Vlk_HMA_B6
aagcaagtataagagtgaa

SEQ ID NO: 25 - DNA, 19 bp, Artificial
  gRNA_B5 sequence of Vlk_HMA_B6
gattcctccaattattttt

SEQ ID NO: 26 - DNA, 19 bp, Artificial
  gRNA_B6 sequence of Vlk_HMA_B6
ccacacccctaaaaataat

SEQ ID NO: 27 - DNA, 25 bp, Artificial
  F_C9 primer sequence
gaccatcctggacttcctgaagagc

SEQ ID NO: 28 - DNA, 27 bp, Artificial
  R_C9 primer sequence
tgcaggtagtacaggtacagcttctcg

SEQ ID NO: 29 - DNA, 19 bp, Artificial
  F_35S primer sequence
gctcctacaaatgccatca

SEQ ID NO: 30 - DNA, 20 bp, Artificial
  R_35S primer sequence
gatagtgggattgtgcgtca

SEQ ID NO: 31 - DNA, 914 bp, Artificial
  PCSs_gDNA sequence of KB108
atggcgatggcgggtttgtatcggcgagttcttccgtcccctccggctgttgatttcgcttctactgaaggaaaggcaagttttagtgctactcttcctttcttccctttgaactgatggttgaagactagggttttagccgtctttgtttgttgttctgttctattttgtcactgatgaatatggattatatcataatgttttaactctttgtttactagctggtaccaatttgttaattggtttggacttcagaggcggattcagaattttaaatctgtagttgtgctttagtattttgccacaatggttgctaactagtataagtatatagttaatgaaatacgcgatatctgcaattcaagctgaacgtaattgatttagtagagtcagcaagatccaggtgaccaaagtgatcagcagcttgatctgctactcagccttagggattggcctatatgatacatgttagataattgtttatcgtaaattgaccaattcgctgtttatcgtaattgtttgtccagaccccatagtgagaactgtggccatggcagacttaaaagaaaatgtgataaaaggaaatcaggaatctgttctgttttacgtgaacttgtaaatttcgctgttcttaagggtttgtagctatttttgtgctttccacatgaagcatctgactttcattttgtactgatttttatgtgtttttttggagctgcagcaacttttcttggaggccatccagaatggaacaatggaaggatttttcaagttgatctcttattttcagacacagtctgaaccggcctattgtggtttggctagcctttccatggtcttgaatgcccttgctattgatccaggaagaaaatggaaaggtaattctactatcatcaagtgaatttgtgttctaa

SEQ ID NO: 32 - DNA, 233 bp, Artificial
  PCSs_CDS sequence of KB108
atggcgatggcgggtttgtatcggcgagttcttccgtcccctccggctgttgatttcgcttctactgaaggaaagcaacttttcttggaggccatccagaatggaacaatggaaggatttttcaagttgatctcttattttcagacacagtctgaaccggcctattgtggtttggctagcctttccatggtcttgaatgcccttgctattgatccaggaagaaaatggaaagg

SEQ ID NO: 33 - protein, 77 aa, Artificial
  PCSs_amino acid sequence of KB108
MAMAGLYRRVLPSPPAVDFASTEGKQLFLEAIQNGTMEGFFKLISYFQTQSEPAYCGLASLSMVLNALAIDPGRKWK

SEQ ID NO: 34 - DNA, 6901 bp, Artificial
  PCSt_gDNA sequence of KB108
atggcgatggcgggtttatatcggcgagttcttccgtcgcctccggctgttgatttcgcttctactgaaggaaaggcaagttttagtgctactctttcctttttcgtttgtgctgagggttaaagactagggttttagccgtctttggttgttgttctattttgtcacggatgaatatggattatatcataatcttttagctatctgtttactagctggtaccgatttgttaattggtttggactttattcagaattttgaatctgtaattgtgtcgttactatttggccacaatggttgctaacttagtataagtatatatattattaaaataattaaactgtgtctgtaattcagctgaaggcaattgatttaatagagtcagcaagatccaggtgaccacagtgatcaacaacttgatcagctggtcagccttagggattggcctatatgatacacgttagataattgtttattgtaagttgatttagacaccaattcactgctaactgtccagaccccataacatatagtaccttttattcaaatttgtccttgatgttccacatggagaactttggccatggcagtcttaaaagaaaatgtgataaaaggaaatcaggaatctgtttctttttgacgcggacttgtaaattttgctgttcttaaggttttgtagctatttttgtgctttccacattggagggcatctgactttcataaattctactccttcctgcttcaatgttttgttgatatatttatttatgatgtacgatattttttttttggcaattaactgtcggtgtctttgtctgtacttacttttacatctacactttttaagtcttaacatcccatttcttgcggtatgcccttaacttccagatactaaaatgtgtgataatattctagtttattgtacttgcttgcccttggtttaggtatcaattcatcttattgttcggttcgactgttaaggtgtacatctgaagttgatgtcattgtacagcctaatacagatttgcttgaatatcttttttctccagtttgatgctgtttcatgaaattaaaatcctttttgtcattaatatgaagagattgaatgcagttttctctggctctttccatacataattttttatgcgtatctttttattaaaaaaagttgtcacataagattttgactttccattgaagttgaccttcagagaggaatagagaaaaggagttatattaaagtatccaagtaaacatatgcttgacatcattcatattctttttactcttctacatatctacagggatggaatactcaggccattaagtctattacaattgcgccaatactctttatttccaagaacttaataagctttttgtgccgaattgctacagtgacattttccccttcaacttctgcacttgattgaaggatgacttttgcctgccatatgtaaaatgatattaatacgagattaatgagttaccttttcccctataggtcaaagcgagaagagctgcattcctcatttgcagccacagcctcattgaggcacatccctttgactccttaccagcccaaacaaccatatgctgcttagctcaattccatctgccttcagttaaattttctgctcagactagccatacccaattcaaatgttgagcaactatttattagtctatcacattgtagccttatacctccaatgaatactcaacataataatactccttggacgactgctggtttcctctgcagatttttatctatgccctcccttcagatcacttgtctggatccttggctcctcccaccctttaagttatgtcattatggaagttgaatcttttaatggggaaagctctcagttattactgcaaccattatcttaaggatctgtttcggtttgctttctcactgattgcctgagtttgatcatattggtttatgataggcttcctagctgaaaaaggggtagctgtccctttcctttttttcccacaatatttcactaggctactgtggtatcgtatggagaacattgtagcttctcttagactgagaaactataagaagataggtgtgccagtgaccgtgtatttgtgcagtgttttgaatttcataggaaacaatgttatttagggagatccttaactttttcggaaaaggttaagtgacccttgcataaacctggaatttgaaggtctgtcttacagagatacctcaaatgcccgcctgatcaaatacaagccattcctacatttatcttgaaatgatactattagatatcaaactaagtcttttgttatatgaactgaatgttatatcaaaaataaatttcttctgtaaacatgaatttgttttcatcagcatagactgcgttttaattagccatacctcagagaaaaaaggaaatgcatgaaacggggtggagggatgagaaagagatacaatatctgatactttgttcagtgactgttctttttgtactgatttttgcgtgttttttggagctgcagcaactttttttggaggccatccagaatggaacaatggaaggatttttcaagttgatctcttattttcagacacagtctgaaccggcctattgtggtttggctagcctttctatggtcttgaatgcccttgctattgatccaggaagaaaatggaaaggtaactctgctatcatcaagtgaatctgtgttcctatccttccggactatatcgatgattttgatcttcactttttcatggccttgccttctgaattgttaataaggtctcaggctctttatatgtttatttccttgatgaaggtctttacatctttattttccaataatttcgcctatcccgacctgcccatgttagttataagatatagcacagagatgtgtagttcactttgttttttattgttgagatctctattaaaaaaaataagttgatgccttgtctgtgagctttgtaagttaatctgatatgttgtgatgcattggcatgagtctgatatcatctgttctcaacatgaatcctagccactgttgctggacaccttccaaagatctaaactgctatatgtgcaacttaatattttttcttacctcatttattgaaggtgtttattctctggataattgttcatcaaagttgccgttctagtgcagttttgcaaacttaaggctttaacagattagacctcggtaatataattctgagaactcctttagagttaatatctgtgtctgtatttcgttccatttgcatttaacagaaaagggttcatgcatctaggagctgttgctctgagttgcctcatgcatattatttgttgatgtccatttttccaaacttcatggtcacatgaaatcattcttattgtcagtttctgtttccaatttacatgaactagaaacatcttagataatattttgtttcaatgttcacatcttaaaaacttgaaaaccttttctttccaccaattaacttatatgttagggtcatatcttggtcaccaatgtctactgatattaaagagtcagtttggaaaagaaggagcttggtgaggacagctttaacgttcttagctttcatcacctgaaactgtttgatgcttgtacgtttctctttcgaacttggttgttcatatcctttttatagtctgcttgcgtcaatgagcttggagtttattatcttatcagcttcaacaaactgtcagggccttggagatggtttgatgaatctatgttggactgttgtgagcctctggagaaggttaaagctaaagggatctcttttgggaaagtggtatgtttggctcactgtgcaggagcgaaggtagaagcttttcgctctaatcatagtactattgatgacttccgtaaacaagtcatggcctgcactactagtgataattgtcatctgatctcatcatatcatagaggcctttttaagcaggtaaatgtaaacatagcttctctatccccatgcttatgattgtgaagaagtatattgctgcatcgggagtttgaccacacaggatctactgaatcttatggcagacaggttcgggccacttttcgcctattggtggttatcacgtgggaaaggatatggcactgattctagatgttgcgaggtttaaatatcctcctcattgggttcccctccctctcctttgggaagccatgaacacaattgatgaagctacaggattacatagggggtatgcacttccagttagctgtgctgtgatattgatattgattttcataatggctatgagttgttgcctgttagcatgctctttaccttgaacagcgtctcgttcatgctttttagctgtcaactgatactggctaagcatttagcttaaggaatataaatatgtagaagaagtagttttactatgtgttttggtcttcctcttataattaagactaaaattggaaattaaatgctgaactctttcggcactgcttctggtcttcttcaaacataactgtagattctcttcattattttctcttcctattctccttctgctcaaatttagcggtccgtctatatccatctcacacttgatatatttttatcattctgcagcatggatttcagtgacttatgtgcaaaaacataattttcaggtttatgctaattactaagcttcacagagctcctgcactgctatataccctggtaaggtttaagtagctgctattatgttattcgctttaaatataaactccttcgaagttcgaacaaactataagaagcaacttctattactgcttgtgagatcctttgctagttgattcttttcagtacaccagcttggtcggatagttgttaactactactttacctaccttttacatgctataacataatgataacatctttgtcaaatgcttgggaattgtggtcttagtgccaggtgattagcttggacttttgtttcaaaactatgatgtcattgcaatatttatggagtttaacaacattggcctttagaactatttaacgtgtaattttttatgtaaattttctataataaagagggaggaatgtactgtattgaattactcctggattttcttctttctgcgactagtcatgtattttgaatggtgaacttctacagagctgtaaacatgagagttgggtcactatctcaaagcatttgatggatgatcttcctgtcctgttaagttctgagaatgtgaagggcataaaagatgttctctctactgttctttcaaatctaccttcaaattttgttgaattcataaagtggatagcggaagttcgaaggcaagaggagaatggtcaaaatttgagtgacgaggagaaaggaaggctagctatcaaggtaaggtatctgaagaaatttagtaaatagaaagaaaaaaaagaaggctcctagaaatggtcatgtagatttgatcgggtaacttgcaggataggtgattatttatgagttcagtaaccactcttaccttactttgcaggaagaggtattgaaacaagtgcaggacactcctctttataagcatgtcacaagcattttattttcaaaaaattctatctgccagtcaaaagcagcatcagacagcagtttggctaatgttgccgcaaacatttgctgccaaggagcaggtctttttgcaggaagatctggttcatcggataggttttgctgtctccaaacatgtgttagatgctacagagctaccgggggcaattctgctacagtggtgtctgggacagttgtaaatgggaatggggagcagggggttgatgttctggtccctacatctctagcaaagactagctgctgtccctcagggcaagctggttgctcgccaatgcaccctgcaagtaacgatgtgctgacagcactattgctggcattacctccacatacatggtctcgaataaaagatacgaaggtcttgcaggaaatagagaaccttgtctcagcagagaacctgcctcctttgctgcaagaagaggtatctttgtacatttgtctcttatcacatttgctttcttgttttaaaattcctctttgttgtttgtgttgggaagcccagaactggaggattgcagtaggttaagggccagcagaaagctaggcaattttatgattaatctaacaatattgaaatattggaaagaaaggagtccaaatagagttaaatggatgtccaggatttatatagcgaccccaactagcttaggattgaggcttttgttttgttgttgtcattttgtattgggaaattcacatgagcacttagaatggccttcataactatatcagagctttctacctcttttctaattggtagttcttattatggcccaatagaagatttttcctgcctaagattatctatttcactgcactgcatattttatgaaaaatagtctaatcctcttgaagcaggttatcatatcataacaggcttaactgatttttcatttctagtctatctgaaggagcatataactgataaatcttatgcataatgagacctaaggcgcataactctgtcttctgcatgttattcagccgtttaatgagtttgtctctcatgctatgcatctttgaccaattaacttagttcaacggtatgagttatgaaattctgagatgtgtgctgggttgatgaactagtagatacgctctgagaatgcattttgttctaatgactacttaaatctttcacttggttaaccattaaacatggtgtaatatacttctgaatatccttttgggcgactgcagattttgcacctgcgaggacagttcctcctcctcaagaaatgcaaggataacaaggtagaagaagatttagctgcacctcccttctagctttgctttcc

SEQ ID NO: 35 - DNA, 1506 bp, Artificial
  PCSt_CDS sequence of KB108
atggcgatggcgggtttatatcggcgagttcttccgtcgcctccggctgttgatttcgcttctactgaaggaaagcaactttttttggaggccatccagaatggaacaatggaaggatttttcaagttgatctcttattttcagacacagtctgaaccggcctattgtggtttggctagcctttctatggtcttgaatgcccttgctattgatccaggaagaaaatggaaagggccttggagatggtttgatgaatctatgttggactgttgtgagcctctggagaaggttaaagctaaagggatctcttttgggaaagtggtatgtttggctcactgtgcaggagcgaaggtagaagcttttcgctctaatcatagtactattgatgacttccgtaaacaagtcatggcctgcactactagtgataattgtcatctgatctcatcatatcatagaggcctttttaagcagacaggttcgggccacttttcgcctattggtggttatcacgtgggaaaggatatggcactgattctagatgttgcgaggtttaaatatcctcctcattgggttcccctccctctcctttgggaagccatgaacacaattgatgaagctacaggattacatagggggtttatgctaattactaagcttcacagagctcctgcactgctatataccctgagctgtaaacatgagagttgggtcactatctcaaagcatttgatggatgatcttcctgtcctgttaagttctgagaatgtgaagggcataaaagatgttctctctactgttctttcaaatctaccttcaaattttgttgaattcataaagtggatagcggaagttcgaaggcaagaggagaatggtcaaaatttgagtgacgaggagaaaggaaggctagctatcaaggaagaggtattgaaacaagtgcaggacactcctctttataagcatgtcacaagcattttattttcaaaaaattctatctgccagtcaaaagcagcatcagacagcagtttggctaatgttgccgcaaacatttgctgccaaggagcaggtctttttgcaggaagatctggttcatcggataggttttgctgtctccaaacatgtgttagatgctacagagctaccgggggcaattctgctacagtggtgtctgggacagttgtaaatgggaatggggagcagggggttgatgttctggtccctacatctctagcaaagactagctgctgtccctcagggcaagctggttgctcgccaatgcaccctgcaagtaacgatgtgctgacagcactattgctggcattacctccacatacatggtctcgaataaaagatacgaaggtcttgcaggaaatagagaaccttgtctcagcagagaacctgcctcctttgctgcaagaagagattttgcacctgcgaggacagttcctcctcctcaagaaatgcaaggataacaaggtagaagaagatttagctgcacctcccttctag

SEQ ID NO: 36 - protein, 501 aa, Artificial
  PCSt_amino acid sequence of KB108
MAMAGLYRRVLPSPPAVDFASTEGKQLFLEAIQNGTMEGFFKLISYFQTQSEPAYCGLASLSMVLNALAIDPGRKWKGPWRWFDESMLDCCEPLEKVKAKGISFGKVVCLAHCAGAKVEAFRSNHSTIDDFRKQVMACTTSDNCHLISSYHRGLFKQTGSGHFSPIGGYHVGKDMALILDVARFKYPPHWVPLPLLWEAMNTIDEATGLHRGFMLITKLHRAPALLYTLSCKHESWVTISKHLMDDLPVLLSSENVKGIKDVLSTVLSNLPSNFVEFIKWIAEVRRQEENGQNLSDEEKGRLAIKEEVLKQVQDTPLYKHVTSILFSKNSICQSKAASDSSLANVAANICCQGAGLFAGRSGSSDRFCCLQTCVRCYRATGGNSATVVSGTVVNGNGEQGVDVLVPTSLAKTSCCPSGQAGCSPMHPASNDVLTALLLALPPHTWSRIKDTKVLQEIENLVSAENLPPLLQEEILHLRGQFLLLKKCKDNKVEEDLAAPPF

SEQ ID NO: 37 - DNA, 915 bp, Artificial
  PCSs_gDNA sequence of PCSst
atggcgatggcgggtttgtatcggcgagttcttccgtcccctccggctgttgatttcgcttctactgaaggaaaggcaagttttagtgctactcttcctttcttccctttgaactgatggttgaagactagggttttagccgtctttgtttgttgttctgttctattttgtcactgatgaatatggattatatcataatgttttaactctttgtttactagctggtaccaatttgttaattggtttggacttcagaggcggattcagaattttaaatctgtagttgtgctttagtattttgccacaatggttgctaactagtataagtatatagttaatgaaatacgcgatatctgcaattcaagctgaacgtaattgatttagtagagtcagcaagatccaggtgaccaaagtgatcagcagcttgatctgctactcagccttagggattggcctatatgatacatgttagataattgtttatcgtaaattgaccaattcgctgtttatcgtaattgtttgtccagaccccatagtgagaactgtggccatggcagacttaaaagaaaatgtgataaaaggaaatcaggaatctgttctgttttacgtgaacttgtaaatttcgctgttcttaagggtttgtagctatttttgtgctttccacatgaagcatctgactttcattttgtactgatttttatgtgtttttttggagctgcagcaacttttcttggaggccatccagaatggaaacaatggaaggatttttcaagttgatctcttattttcagacacagtctgaaccggcctattgtggtttggctagcctttccatggtcttgaatgcccttgctattgatccaggaagaaaatggaaaggtaattctactatcatcaagtgaatttgtgttctaa

SEQ ID NO: 38 - DNA, 234 bp, Artificial
  PCSs_CDS sequence of PCSst
atggcgatggcgggtttgtatcggcgagttcttccgtcccctccggctgttgatttcgcttctactgaaggaaagcaacttttcttggaggccatccagaatggaaacaatggaaggatttttcaagttgatctcttattttcagacacagtctgaaccggcctattgtggtttggctagcctttccatggtcttgaatgcccttgctattgatccaggaagaaaatggaaagg

SEQ ID NO: 39 - protein, 78 aa, Artificial
  PCSs_amino acid sequence of PCSst
  SITE (52)..(52): X or Xaa is * which is the result of early stop codon
  misc_feature (52)..(52): Xaa can be any naturally occurring amino acid
  SITE (60)..(60): X or Xaa is * which is the result of early stop codon
  misc_feature (60)..(60): Xaa can be any naturally occurring amino acid
  SITE (71)..(71): X or Xaa is * which is the result of early stop codon
  misc_feature (71)..(71): Xaa can be any naturally occurring amino acid
MAMAGLYRRVLPSPPAVDFASTEGKQLFLEAIQNGNNGRIFQVDLLFSDTVXTGLLWFGXPFHGLECPCYXSRKKMER

SEQ ID NO: 40 - DNA, 6902 bp, Artificial
  PCSt_gDNA sequence of PCSst
atggcgatggcgggtttatatcggcgagttcttccgtcgcctccggctgttgatttcgcttctactgaaggaaaggcaagttttagtgctactctttcctttttcgtttgtgctgagggttaaagactagggttttagccgtctttggttgttgttctattttgtcacggatgaatatggattatatcataatcttttagctatctgtttactagctggtaccgatttgttaattggtttggactttattcagaattttgaatctgtaattgtgtcgttactatttggccacaatggttgctaacttagtataagtatatatattattaaaataattaaactgtgtctgtaattcagctgaaggcaattgatttaatagagtcagcaagatccaggtgaccacagtgatcaacaacttgatcagctggtcagccttagggattggcctatatgatacacgttagataattgtttattgtaagttgatttagacaccaattcactgctaactgtccagaccccataacatatagtaccttttattcaaatttgtccttgatgttccacatggagaactttggccatggcagtcttaaaagaaaatgtgataaaaggaaatcaggaatctgtttctttttgacgcggacttgtaaattttgctgttcttaaggttttgtagctatttttgtgctttccacattggagggcatctgactttcataaattctactccttcctgcttcaatgttttgttgatatatttatttatgatgtacgatattttttttttggcaattaactgtcggtgtctttgtctgtacttacttttacatctacactttttaagtcttaacatcccatttcttgcggtatgcccttaacttccagatactaaaatgtgtgataatattctagtttattgtacttgcttgcccttggtttaggtatcaattcatcttattgttcggttcgactgttaaggtgtacatctgaagttgatgtcattgtacagcctaatacagatttgcttgaatatcttttttctccagtttgatgctgtttcatgaaattaaaatcctttttgtcattaatatgaagagattgaatgcagttttctctggctctttccatacataattttttatgcgtatctttttattaaaaaaagttgtcacataagattttgactttccattgaagttgaccttcagagaggaatagagaaaaggagttatattaaagtatccaagtaaacatatgcttgacatcattcatattctttttactcttctacatatctacagggatggaatactcaggccattaagtctattacaattgcgccaatactctttatttccaagaacttaataagctttttgtgccgaattgctacagtgacattttccccttcaacttctgcacttgattgaaggatgacttttgcctgccatatgtaaaatgatattaatacgagattaatgagttaccttttcccctataggtcaaagcgagaagagctgcattcctcatttgcagccacagcctcattgaggcacatccctttgactccttaccagcccaaacaaccatatgctgcttagctcaattccatctgccttcagttaaattttctgctcagactagccatacccaattcaaatgttgagcaactatttattagtctatcacattgtagccttatacctccaatgaatactcaacataataatactccttggacgactgctggtttcctctgcagatttttatctatgccctcccttcagatcacttgtctggatccttggctcctcccaccctttaagttatgtcattatggaagttgaatcttttaatggggaaagctctcagttattactgcaaccattatcttaaggatctgtttcggtttgctttctcactgattgcctgagtttgatcatattggtttatgataggcttcctagctgaaaaaggggtagctgtccctttcctttttttcccacaatatttcactaggctactgtggtatcgtatggagaacattgtagcttctcttagactgagaaactataagaagataggtgtgccagtgaccgtgtatttgtgcagtgttttgaatttcataggaaacaatgttatttagggagatccttaactttttcggaaaaggttaagtgacccttgcataaacctggaatttgaaggtctgtcttacagagatacctcaaatgcccgcctgatcaaatacaagccattcctacatttatcttgaaatgatactattagatatcaaactaagtcttttgttatatgaactgaatgttatatcaaaaataaatttcttctgtaaacatgaatttgttttcatcagcatagactgcgttttaattagccatacctcagagaaaaaaggaaatgcatgaaacggggtggagggatgagaaagagatacaatatctgatactttgttcagtgactgttctttttgtactgatttttgcgtgttttttggagctgcagcaactttttttggaggccatccagaatggaaacaatggaaggatttttcaagttgatctcttattttcagacacagtctgaaccggcctattgtggtttggctagcctttctatggtcttgaatgcccttgctattgatccaggaagaaaatggaaaggtaactctgctatcatcaagtgaatctgtgttcctatccttccggactatatcgatgattttgatcttcactttttcatggccttgccttctgaattgttaataaggtctcaggctctttatatgtttatttccttgatgaaggtctttacatctttattttccaataatttcgcctatcccgacctgcccatgttagttataagatatagcacagagatgtgtagttcactttgttttttattgttgagatctctattaaaaaaaataagttgatgccttgtctgtgagctttgtaagttaatctgatatgttgtgatgcattggcatgagtctgatatcatctgttctcaacatgaatcctagccactgttgctggacaccttccaaagatctaaactgctatatgtgcaacttaatattttttcttacctcatttattgaaggtgtttattctctggataattgttcatcaaagttgccgttctagtgcagttttgcaaacttaaggctttaacagattagacctcggtaatataattctgagaactcctttagagttaatatctgtgtctgtatttcgttccatttgcatttaacagaaaagggttcatgcatctaggagctgttgctctgagttgcctcatgcatattatttgttgatgtccatttttccaaacttcatggtcacatgaaatcattcttattgtcagtttctgtttccaatttacatgaactagaaacatcttagataatattttgtttcaatgttcacatcttaaaaacttgaaaaccttttctttccaccaattaacttatatgttagggtcatatcttggtcaccaatgtctactgatattaaagagtcagtttggaaaagaaggagcttggtgaggacagctttaacgttcttagctttcatcacctgaaactgtttgatgcttgtacgtttctctttcgaacttggttgttcatatcctttttatagtctgcttgcgtcaatgagcttggagtttattatcttatcagcttcaacaaactgtcagggccttggagatggtttgatgaatctatgttggactgttgtgagcctctggagaaggttaaagctaaagggatctcttttgggaaagtggtatgtttggctcactgtgcaggagcgaaggtagaagcttttcgctctaatcatagtactattgatgacttccgtaaacaagtcatggcctgcactactagtgataattgtcatctgatctcatcatatcatagaggcctttttaagcaggtaaatgtaaacatagcttctctatccccatgcttatgattgtgaagaagtatattgctgcatcgggagtttgaccacacaggatctactgaatcttatggcagacaggttcgggccacttttcgcctattggtggttatcacgtgggaaaggatatggcactgattctagatgttgcgaggtttaaatatcctcctcattgggttcccctccctctcctttgggaagccatgaacacaattgatgaagctacaggattacatagggggtatgcacttccagttagctgtgctgtgatattgatattgattttcataatggctatgagttgttgcctgttagcatgctctttaccttgaacagcgtctcgttcatgctttttagctgtcaactgatactggctaagcatttagcttaaggaatataaatatgtagaagaagtagttttactatgtgttttggtcttcctcttataattaagactaaaattggaaattaaatgctgaactctttcggcactgcttctggtcttcttcaaacataactgtagattctcttcattattttctcttcctattctccttctgctcaaatttagcggtccgtctatatccatctcacacttgatatatttttatcattctgcagcatggatttcagtgacttatgtgcaaaaacataattttcaggtttatgctaattactaagcttcacagagctcctgcactgctatataccctggtaaggtttaagtagctgctattatgttattcgctttaaatataaactccttcgaagttcgaacaaactataagaagcaacttctattactgcttgtgagatcctttgctagttgattcttttcagtacaccagcttggtcggatagttgttaactactactttacctaccttttacatgctataacataatgataacatctttgtcaaatgcttgggaattgtggtcttagtgccaggtgattagcttggacttttgtttcaaaactatgatgtcattgcaatatttatggagtttaacaacattggcctttagaactatttaacgtgtaattttttatgtaaattttctataataaagagggaggaatgtactgtattgaattactcctggattttcttctttctgcgactagtcatgtattttgaatggtgaacttctacagagctgtaaacatgagagttgggtcactatctcaaagcatttgatggatgatcttcctgtcctgttaagttctgagaatgtgaagggcataaaagatgttctctctactgttctttcaaatctaccttcaaattttgttgaattcataaagtggatagcggaagttcgaaggcaagaggagaatggtcaaaatttgagtgacgaggagaaaggaaggctagctatcaaggtaaggtatctgaagaaatttagtaaatagaaagaaaaaaaagaaggctcctagaaatggtcatgtagatttgatcgggtaacttgcaggataggtgattatttatgagttcagtaaccactcttaccttactttgcaggaagaggtattgaaacaagtgcaggacactcctctttataagcatgtcacaagcattttattttcaaaaaattctatctgccagtcaaaagcagcatcagacagcagtttggctaatgttgccgcaaacatttgctgccaaggagcaggtctttttgcaggaagatctggttcatcggataggttttgctgtctccaaacatgtgttagatgctacagagctaccgggggcaattctgctacagtggtgtctgggacagttgtaaatgggaatggggagcagggggttgatgttctggtccctacatctctagcaaagactagctgctgtccctcagggcaagctggttgctcgccaatgcaccctgcaagtaacgatgtgctgacagcactattgctggcattacctccacatacatggtctcgaataaaagatacgaaggtcttgcaggaaatagagaaccttgtctcagcagagaacctgcctcctttgctgcaagaagaggtatctttgtacatttgtctcttatcacatttgctttcttgttttaaaattcctctttgttgtttgtgttgggaagcccagaactggaggattgcagtaggttaagggccagcagaaagctaggcaattttatgattaatctaacaatattgaaatattggaaagaaaggagtccaaatagagttaaatggatgtccaggatttatatagcgaccccaactagcttaggattgaggcttttgttttgttgttgtcattttgtattgggaaattcacatgagcacttagaatggccttcataactatatcagagctttctacctcttttctaattggtagttcttattatggcccaatagaagatttttcctgcctaagattatctatttcactgcactgcatattttatgaaaaatagtctaatcctcttgaagcaggttatcatatcataacaggcttaactgatttttcatttctagtctatctgaaggagcatataactgataaatcttatgcataatgagacctaaggcgcataactctgtcttctgcatgttattcagccgtttaatgagtttgtctctcatgctatgcatctttgaccaattaacttagttcaacggtatgagttatgaaattctgagatgtgtgctgggttgatgaactagtagatacgctctgagaatgcattttgttctaatgactacttaaatctttcacttggttaaccattaaacatggtgtaatatacttctgaatatccttttgggcgactgcagattttgcacctgcgaggacagttcctcctcctcaagaaatgcaaggataacaaggtagaagaagatttagctgcacctcccttctagctttgctttcc

SEQ ID NO: 41 - DNA, 1507 bp, Artificial
  PCSt_CDS sequence of PCSst
atggcgatggcgggtttatatcggcgagttcttccgtcgcctccggctgttgatttcgcttctactgaaggaaagcaactttttttggaggccatccagaatggaaacaatggaaggatttttcaagttgatctcttattttcagacacagtctgaaccggcctattgtggtttggctagcctttctatggtcttgaatgcccttgctattgatccaggaagaaaatggaaagggccttggagatggtttgatgaatctatgttggactgttgtgagcctctggagaaggttaaagctaaagggatctcttttgggaaagtggtatgtttggctcactgtgcaggagcgaaggtagaagcttttcgctctaatcatagtactattgatgacttccgtaaacaagtcatggcctgcactactagtgataattgtcatctgatctcatcatatcatagaggcctttttaagcagacaggttcgggccacttttcgcctattggtggttatcacgtgggaaaggatatggcactgattctagatgttgcgaggtttaaatatcctcctcattgggttcccctccctctcctttgggaagccatgaacacaattgatgaagctacaggattacatagggggtttatgctaattactaagcttcacagagctcctgcactgctatataccctgagctgtaaacatgagagttgggtcactatctcaaagcatttgatggatgatcttcctgtcctgttaagttctgagaatgtgaagggcataaaagatgttctctctactgttctttcaaatctaccttcaaattttgttgaattcataaagtggatagcggaagttcgaaggcaagaggagaatggtcaaaatttgagtgacgaggagaaaggaaggctagctatcaaggaagaggtattgaaacaagtgcaggacactcctctttataagcatgtcacaagcattttattttcaaaaaattctatctgccagtcaaaagcagcatcagacagcagtttggctaatgttgccgcaaacatttgctgccaaggagcaggtctttttgcaggaagatctggttcatcggataggttttgctgtctccaaacatgtgttagatgctacagagctaccgggggcaattctgctacagtggtgtctgggacagttgtaaatgggaatggggagcagggggttgatgttctggtccctacatctctagcaaagactagctgctgtccctcagggcaagctggttgctcgccaatgcaccctgcaagtaacgatgtgctgacagcactattgctggcattacctccacatacatggtctcgaataaaagatacgaaggtcttgcaggaaatagagaaccttgtctcagcagagaacctgcctcctttgctgcaagaagagattttgcacctgcgaggacagttcctcctcctcaagaaatgcaaggataacaaggtagaagaagatttagctgcacctcccttctag

SEQ ID NO: 42 - protein, 502 aa, Artificial
  PCSt_amino acid sequence of PCSst
  SITE (52)..(52): X or Xaa is * which is the result of early stop codon
  misc_feature (52)..(52): Xaa can be any naturally occurring amino acid
  SITE (60)..(60): X or Xaa is * which is the result of early stop codon
  misc_feature (60)..(60): Xaa can be any naturally occurring amino acid
  SITE (71)..(71): X or Xaa is * which is the result of early stop codon
  misc_feature (71)..(71): Xaa can be any naturally occurring amino acid
  SITE (84)..(85): X or Xaa is * which is the result of early stop codon
  misc_feature (84)..(85): Xaa can be any naturally occurring amino acid
  SITE (92)..(92): X or Xaa is * which is the result of early stop codon
  misc_feature (92)..(92): Xaa can be any naturally occurring amino acid
  SITE (98)..(98): X or Xaa is * which is the result of early stop codon
  misc_feature (98)..(98): Xaa can be any naturally occurring amino acid
  SITE (100)..(100): X or Xaa is * which is the result of early stop codon
  misc_feature (100)..(100): Xaa can be any naturally occurring amino acid
  SITE (124)..(124): X or Xaa is * which is the result of early stop codon
  misc_feature (124)..(124): Xaa can be any naturally occurring amino acid
  SITE (126)..(126): X or Xaa is * which is the result of early stop codon
  misc_feature (126)..(126): Xaa can be any naturally occurring amino acid
  SITE (129)..(130): X or Xaa is * which is the result of early stop codon
  misc_feature (129)..(130): Xaa can be any naturally occurring amino acid
  SITE (133)..(133): X or Xaa is * which is the result of early stop codon
  misc_feature (133)..(133): Xaa can be any naturally occurring amino acid
  SITE (141)..(143): X or Xaa is * which is the result of early stop codon
  misc_feature (141)..(143): Xaa can be any naturally occurring amino acid
  SITE (152)..(152): X or Xaa is * which is the result of early stop codon
  misc_feature (152)..(152): Xaa can be any naturally occurring amino acid
  SITE (156)..(156): X or Xaa is * which is the result of early stop codon
  misc_feature (156)..(156): Xaa can be any naturally occurring amino acid
  SITE (185)..(185): X or Xaa is * which is the result of early stop codon
  misc_feature (185)..(185): Xaa can be any naturally occurring amino acid
  SITE (204)..(205): X or Xaa is * which is the result of early stop codon
  misc_feature (204)..(205): Xaa can be any naturally occurring amino acid
  SITE (211)..(211): X or Xaa is * which is the result of early stop codon
  misc_feature (211)..(211): Xaa can be any naturally occurring amino acid
  SITE (218)..(218): X or Xaa is * which is the result of early stop codon
  misc_feature (218)..(218): Xaa can be any naturally occurring amino acid
  SITE (232)..(232): X or Xaa is * which is the result of early stop codon
  misc_feature (232)..(232): Xaa can be any naturally occurring amino acid
  SITE (234)..(234): X or Xaa is * which is the result of early stop codon
  misc_feature (234)..(234): Xaa can be any naturally occurring amino acid
  SITE (246)..(246): X or Xaa is * which is the result of early stop codon
  misc_feature (246)..(246): Xaa can be any naturally occurring amino acid
  SITE (254)..(254): X or Xaa is * which is the result of early stop codon
  misc_feature (254)..(254): Xaa can be any naturally occurring amino acid
  SITE (276)..(276): X or Xaa is * which is the result of early stop codon
  misc_feature (276)..(276): Xaa can be any naturally occurring amino acid
  SITE (296)..(296): X or Xaa is * which is the result of early stop codon
  misc_feature (296)..(296): Xaa can be any naturally occurring amino acid
  SITE (319)..(319): X or Xaa is * which is the result of early stop codon
  misc_feature (319)..(319): Xaa can be any naturally occurring amino acid
  SITE (344)..(344): X or Xaa is * which is the result of early stop codon
  misc_feature (344)..(344): Xaa can be any naturally occurring amino acid
  SITE (366)..(366): X or Xaa is * which is the result of early stop codon
  misc_feature (366)..(366): Xaa can be any naturally occurring amino acid
  SITE (375)..(375): X or Xaa is * which is the result of early stop codon
  misc_feature (375)..(375): Xaa can be any naturally occurring amino acid
  SITE (402)..(402): X or Xaa is * which is the result of early stop codon
  misc_feature (402)..(402): Xaa can be any naturally occurring amino acid
  SITE (413)..(413): X or Xaa is * which is the result of early stop codon
  misc_feature (413)..(413): Xaa can be any naturally occurring amino acid
  SITE (430)..(430): X or Xaa is * which is the result of early stop codon
  misc_feature (430)..(430): Xaa can be any naturally occurring amino acid
  SITE (490)..(490): X or Xaa is * which is the result of early stop codon
  misc_feature (490)..(490): Xaa can be any naturally occurring amino acid
MAMAGLYRRVLPSPPAVDFASTEGKQLFLEAIQNGNNGRIFQVDLLFSDTVXTGLLWFGXPFYGLECPCYXSRKKMERALEMVXXIYVGLLXASGEGXSXRDLFWESGMFGSLCRSEGRSFSLXSXYYXXLPXTSHGLHYXXXLSSDLIISXRPFXADRFGPLFAYWWLSRGKGYGTDSRCCEVXISSSLGSPPSPLGSHEHNXXSYRITXGVYANYXASQSSCTAIYPELXTXELGHYLKAFDGXSSCPVKFXECEGHKRCSLYCSFKSTFKFCXIHKVDSGSSKARGEWSKFEXRGERKASYQGRGIETSAGHSSLXACHKHFIFKKFYLPVKSSIRQQFGXCCRKHLLPRSRSFCRKIWFIGXVLLSPNMCXMLQSYRGQFCYSGVWDSCKWEWGAGGXCSGPYISSKDXLLSLRASWLLANAPCKXRCADSTIAGITSTYMVSNKRYEGLAGNREPCLSREPASFAARRDFAPARTVPPPQEMQGXQGRRRFSCTSLL

SEQ ID NO: 43 - DNA, 304 bp, Artificial
  P_U6 sequence
agaaatctcaaaattccggcagaacaattttgaatctcgatccgtagaaacgagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgatt

(19) United States
(12) Patent Application Publication
(10) Pub. No.: US 2024/0052360 A1
(43) Pub. Date: Feb. 15, 2024

(54) TRANSGENIC PLANT WITH REDUCED HEAVY METALS AND METHODS FOR PREPARATION THEREOF

(71) Applicant: KT&G CORPORATION, Daejeon (KR)

(72) Inventors: Hyo Seok SEO, Sejong (KR); Young Gi LEE, Daejeon (KR); Jeong Heon LEE, Daejeon (KR); Kwang Chul KIM, Daejeon (KR); Woong Hyun NA, Daejeon (KR); Kyoung Hwan OH, Cheongju-si (KR); Eun Young JEON, Sejong (KR)

(73) Assignee: KT&G CORPORATION, Daejeon (KR)

(21) Appl. No.: 17/642,050

(22) PCT Filed: Aug. 19, 2021
(86) PCT No.: PCT/KR2021/011059

§ 371 Date: Mar. 10, 2022

(30) Foreign Application Priority Data
Sep. 8, 2020 (KR) 10-2020-0114891

(51) Int. Cl. C12N 15/82; C12N 9/10; C12N 9/22; C12N 15/11

(52) U.S. Cl. CPC C12N 15/8271; C12N 9/104; C12Y 203/02015; C12N 9/22; C12N 15/11; C12N 2310/20

(57) ABSTRACT

Provided are: a plant cell, in which expression or activity of a phytochelatin synthase (PCS) gene or a protein encoded by the PCS gene is reduced as compared with a parent cell; a plant having that has reduced heavy metal absorption, and includes the plant cell; a method of reducing heavy metals in a plant, the method including reducing expression or activity of a PCS gene or a protein encoded by the PCS gene, as compared with a parent cell; a CRISPR-Cas9 recombinant vector including a single guide RNA targeting a PCS gene; and a method of preparing a plant with reduced heavy metals, the method including transforming a plant cell with the recombinant vector.

### TECHNICAL FIELD

The present disclosure relates to a transgenic plant with reduced heavy metals and a method for preparation thereof

### BACKGROUND ART

Heavy metals such as lead, cadmium, mercury, etc. are pollutants derived from the environment, and exposed through foods, water, air, soil, etc. The general public mainly intakes heavy metals through foods, and trace amounts of heavy metals absorbed into the body accumulate in organs such as bones, kidneys, etc., and are known to adversely affect health. The Ministry of Food and Drug Safety (MFDS) tracked changes in the concentration of heavy metals in the bodies of Koreans from 2010 to 2015 and analyzed the relationship with food intake or lifestyle. As a result, it was confirmed that drinking, smoking, and eating habits are related to the concentration of heavy metals in the body. With regard to smoking, the absorption rate through inhalation is higher than the absorption rate through ingestion, and harmful substances such as heavy metals from cigarettes themselves enter the body and increase the concentration of heavy metals in the body. Therefore, it is required to develop tobacco plants in which the heavy metal content therein or the absorption rate of heavy metals from soil during cultivation may be reduced.

In addition, many heavy metals naturally exist in the soil and are absorbed by plants to different degrees. Some heavy metals, such as manganese or zinc, are essential for plants because they are cofactors required for enzymatic activity, whereas other heavy metals are not essential for plants, and thus it is beneficial to reduce the concentration of heavy metals. For example, cadmium (Cd) has been reported as a heavy metal having no beneficial effect on plant or human development, and is classified as a human carcinogen. When Cd excessively accumulates in plants, it has detrimental effects such as the reduction of leaf surface and the reduction of dry weight, water content, chlorophyll content, and carotenoid content.

In particular, tobacco is a plant species characterized by having an ability to accumulate four times higher levels of Cd in the shoot than in the root, and thus it is desirable to reduce the accumulation of Cd in plants such as tobacco. In this regard, there is known a method of preparing a plant variant having reduced expression or activity of an NtHMA gene as a tobacco plant with reduced cadmium accumulation (Korean Patent Publication No. 10-2018-0107123). However, the tobacco plant variant prepared by the method has a problem in that it cannot grow normally as compared with the wild-type tobacco plant.

In view of this background, the present inventors found that by inducing a mutation in a phytochelatin synthase (NtPCS) gene of tobacco plants, the content or the absorption of heavy metals is remarkably reduced as compared with wild-type tobacco plants, while maintaining excellent growth characteristics, thereby completing the present disclosure.

### DESCRIPTION OF EMBODIMENTS

### Technical Problem

An aspect provides a plant cell, in which expression or activity of a phytochelatin synthase (PCS) gene or a protein encoded by the PCS gene is reduced, as compared with a parent cell.

Another aspect provides a plant having reduced heavy metal absorption, the plant including a plant cell, in which expression or activity of a PCS gene or a protein encoded by the PCS gene is reduced, as compared with a parent cell.

Still another aspect provides a method of reducing heavy metals in a plant, the method including reducing expression or activity of a PCS gene or a protein encoded by the PCS gene, as compared with a parent cell.

Still another aspect provides a CRISPR-Cas9 recombinant vector including a single guide RNA targeting a PCS gene.

Still another aspect provides a method of preparing a plant having reduced heavy metal absorption, the method including transforming a plant cell with a CRISPR-Cas9 recombinant vector including a single guide RNA targeting a PCS gene.

### Solution to Problem

An aspect provides a plant cell, in which expression or activity of a phytochelatin synthase (PCS) gene or a protein encoded by the PCS gene is reduced, as compared with a parent cell.

As used herein, the term “parent cell”, which is a cell that has not been artificially manipulated to reduce expression or activity of the PCS gene or the protein encoded by the PCS gene, may refer to a cell freshly isolated from a plant or a cell cultured therefrom.

As used herein, the term “phytochelatin synthase (PCS)” is, also called glutathione gamma-glutamylcysteinyltransferase or gamma-glutamylcysteine dipeptidyl transpeptidase, an enzyme that produces two products of Gly and [Glu(-Cys)]n+1-Gly from two substrates of glutathione and [Glu(-Cys)]n-Gly. In tobacco plants, PCS is a protein involved in storage and transport of intracellular metal ions by binding thereto, and specifically, it is a protein expected to be involved in the intracellular long-distance transport of heavy metals including cadmium, zinc, etc.

The PCS gene may be a PCS gene (NtPCSs) derived from Nicotiana sylvestris, a PCS gene (NtPCSt) derived from Nicotiana tomentosiformis, or a combination thereof (NtPCSst).

The plant cell may be genetically engineered to reduce expression or activity of the PCS gene or the protein encoded by the PCS gene. As used herein, the term “genetic engineering” or “genetically engineered” may refer to manipulation to introduce one or more genetic modifications into a cell or a cell prepared thereby.

The genetic engineering may be induced by modification in a nucleotide sequence of the PCS gene by a physical method. The physical method may be, for example, X-ray irradiation, gamma-ray irradiation, etc.

The genetic engineering may be induced by a modification in the nucleotide sequence of the PCS gene or a change in the gene expression by a chemical method. The chemical method may be, for example, ethyl methanesulfonate treatment, dimethyl sulfate treatment, etc.

The genetic engineering may be induced by a modification in the nucleotide sequence of the PCS gene by a gene editing system. The gene editing system may be, for example, a meganuclease system, a zinc finger nuclease system, a transcription activator-like effector nuclease (TALEN) system, a CRISPR/Cas9 system, etc. For example, the genetic engineering may be induced by a change in the gene expression by binding to mRNA transcribed from the PCS gene by an RNA interference (RNAi) system.

In one specific embodiment, the plant cell may reduce the expression or activity of the PCS gene or the protein encoded by the PCS gene by the CRISPR/Cas9 system.

The term “genetically engineered plant cell”, in which “the expression or activity of the PCS gene or the protein encoded by the PCS gene is reduced”, the PCS gene is “inactivated”, “the expression or activity” of the PCS protein is reduced, or the PCS gene is “inactivated”, means that the expression or activity level of the PCS gene or the protein encoded by the PCS gene is lower than that of a PCS gene or a protein encoded by a PCS gene, which is measured in a comparable plant cell of the same species or a parent cell thereof, or the PCS gene or the protein encoded by the PCS gene exhibits no expression or activity. In other words, in the plant cell, the expression or activity of the PCS gene or the protein encoded by the PCS gene exhibits about 20% or more, about 30% or more, about 40% or more, about 50% or more, about 55% or more, about 60% or more, about 70% or more, about 75% or more, about 80% or more, about 85% or more, about 90% or more, about 95% or more, or about 100% reduction, as compared with that of the non-engineered original plant cell. The genetically engineered plant cell, in which the expression or activity of the PCS gene or the protein encoded by the PCS gene is reduced, may be identified by any known method in the art. The term “inactivation” may mean that a gene is not expressed at all or a protein having no activity is produced even though expressed. The term “reduction (depression)” may mean that the PCS gene is expressed at a low level, as compared with that of a non-engineered plant cell, or may mean that the activity of the protein encoded by the PCS gene is low even though expressed.

Another aspect relates to a plant having reduced heavy metal absorption, the plant including the plant cell. Detailed descriptions of the plant cell, etc. are as described above.

The plant may be Nicotiana tabacum, specifically, flue—cured—, Burley, old tobacco variety, black, or oriental variety, and more specifically, Burley or flue—cured variety.

In one specific embodiment, the plant may be a plant, in which the content or absorption of heavy metals is reduced in the same generation as well as in subsequent generations, by including the plant cell, in which the expression or activity of the PCS gene or the protein encoded by the PCS gene is reduced. Thus, it is possible to continuously obtain a plant with reduced heavy metals.

Still another aspect provides a method of reducing heavy metals in a plant, the method including reducing expression or activity of the PCS gene or the protein encoded by the PCS gene, as compared with a parent cell of the plant. Detailed descriptions of the parent cell, PCS, plant cell, reduction of the expression or activity, etc. are as described above.

Reducing of the expression or activity of the PCS gene or the PCS protein may be performed by variation, substitution, or deletion of a part or all of the gene encoding the PCS, or insertion of one or more bases in the gene, and may be performed via a PCS gene editing means.

In reducing the expression or activity of the PCS gene or the protein encoded by the PCS gene, at least one selected from the group consisting of an RNA interference (RNAi) system, a meganuclease system, a zinc finger nuclease system, a transcription activator-like effector nuclease (TALEN) system, a CRISPR/Cas9 system, X-ray irradiation, gamma-ray irradiation, ethyl methanesulfonate treatment, and dimethyl sulfate treatment may be performed.

Gene manipulation artificially performed to reduce the expression or activity of the PCS gene or the protein encoded by the PCS gene may be preventing the PCS gene from being expressed into a protein form having its intrinsic function. The gene manipulation may be induced by one or more of the following manipulations: 1) deletion of all or a part of the PCS gene, e.g., deletion of 1 bp or more nucleotides, e.g., 1 nucleotide to 30 nucleotides, 1 nucleotide to 27 nucleotides, 1 nucleotide to 25 nucleotides, 1 nucleotide to 23 nucleotides, 1 nucleotide to 20 nucleotides, 1 nucleotide to 15 nucleotides, 1 nucleotide to 10 nucleotides, 1 to 5 nucleotides, 1 nucleotide to 3 nucleotides, or 1 nucleotide of the PCS gene; 2) substitution of 1 bp or more nucleotides of the PCS gene, e.g., 1 nucleotide to 30 nucleotides, 1 nucleotide to 27 nucleotides, 1 nucleotide to 25 nucleotides, 1 nucleotide to 23 nucleotides, 1 nucleotide to 20 nucleotides, 1 nucleotide to 15 nucleotides, 1 nucleotide to 10 nucleotides, 1 to 5 nucleotides, 1 nucleotide to 3 nucleotides, or 1 nucleotide with different nucleotides from the original (wild-type); 3) insertion of one or more nucleotides, e.g., 1 nucleotide to 30 nucleotides, 1 nucleotide to 27 nucleotides, 1 nucleotide to 25 nucleotides, 1 nucleotide to 23 nucleotides, 1 nucleotide to 20 nucleotides, 1 nucleotide to 15 nucleotides, 1 nucleotide to 10 nucleotides, 1 to 5 nucleotides, 1 nucleotide to 3 nucleotides, or 1 nucleotide (each independently selected from A, T, C, and G) into any site of the target gene; and 4) a combination of two or more selected from 1) to 3).

A part modified in the PCS gene (‘target site’) may be a consecutive nucleotide sequence of 1 bp or more, 3 bp or more, 5 bp or more, 7 bp or more, 10 bp or more, 12 bp or more, 15 bp or more, 17 bp or more, 20 bp or more, e.g., 1 bp to 30 bp, 3 bp to 30 bp, 5 bp to 30 bp, 7 bp to 30 bp, 10 bp to 30 bp, 12 bp to 30 bp, 15 bp to 30 bp, 17 bp to 30 bp, 20 bp to 30 bp, 1 bp to 27 bp, 3 bp to 27 bp, 5 bp to 27 bp, 7 bp to 27 bp, 10 bp to 27 bp, 12 bp to 27 bp, 15 bp to 27 bp, 17 bp to 27 bp, 20 bp to 27 bp, 1 bp to 25 bp, 3 bp to 25 bp, 5 bp to 25 bp, 7 bp to 25 bp, 10 bp to 25 bp, 12 bp to 25 bp, 15 bp to 25 bp, 17 bp to 25 bp, 20 bp to 25 bp, 1 bp to 23 bp, 3 bp to 23 bp, 5 bp to 23 bp, 7 bp to 23 bp, 10 bp to 23 bp, 12 bp to 23 bp, 15 bp to 23 bp, 17 bp to 23 bp, 20 bp to 23 bp, 1 bp to 20 bp, 3 bp to 20 bp, 5 bp to 20 bp, 7 bp to 20 bp, 10 bp to 20 bp, 12 bp to 20 bp, 15 bp to 20 bp, 17 bp to 20 bp, 21 bp to 25 bp, 18 bp to 22 bp, or 21 bp to 23 bp in the gene.

For example, to prepare a PCS-knockout plant cell, single-stranded or double-stranded cleavage of a specific site within a target gene is catalyzed by a genome editing system including a rare-cutting endonuclease that cuts a rare gene sequence with a very low abundance in the genome to reduce expression of the PCS gene which is the target gene. Nucleic acid strand breaks catalyzed by the rare-cutting endonuclease may be repaired through mechanisms such as homologous recombination, non-homologous end joining (NHEJ), etc. The rare-cutting endonuclease may be one or more selected from the group consisting of meganuclease, zinc finger nuclease, CRISPR/Cas9 (Cas9 protein), CRISPR-Cpf1 (Cpf1 protein), and TALE-nuclease.

The heavy metal may be one or more selected from the group consisting of cadmium, arsenic, antimony, lead, mercury, chromium, tin, zinc, barium, bismuth, nickel, cobalt, manganese, iron, copper, and vanadium. In one specific embodiment, the content or absorption of cadmium in the plant may be reduced by reducing the expression or activity of the PCS gene or the protein encoded by the PCS gene.

Still another aspect provides a CRISPR-Cas9 recombinant vector including a single guide RNA targeting the PCS gene. Detailed descriptions of the PCS gene, plant cell, etc. are as described above.

As used herein, the terms “single guide RNA (sgRNA)” may be used interchangeably with “chimeric RNA”, “chimeric guide RNA”, “guide RNA”, “single guide RNA (sgRNA)”, and “synthetic guide RNA”. It refers to a polynucleotide sequence including a guide sequence, a tracr sequence, and/or a tracr mate sequence. The term “guide sequence” refers to an about 20 bp-sequence in a guide RNA that designates a target site, and may be used interchangeably with the term “guide” or “spacer”. Further, the term “tracr mate sequence” may be used interchangeably with the term “direct repeat(s)”. The guide RNA may consist of two RNAs, i.e., CRISPR RNA (crRNA) and transactivating crRNA (tracrRNA), or may be a single-stranded RNA (sgRNA) including a portion of crRNA and tracrRNA and hybridizing with the target DNA.

The PSC gene targeted by the sgRNA may be a PCS gene (NtPCSs) derived from Nicotiana sylvestris, or a PCS gene (NtPCSt) derived from Nicotiana tomentosiformis.

The sgRNA targeting the NtPCSt gene may consist of a nucleotide sequence of SEQ ID NO: 1 or SEQ ID NO: 2, the sgRNA targeting the NtPCSt gene may consist of a nucleotide sequence of SEQ ID NO: 3 or SEQ ID NO: 4, and the sgRNA targeting both the NtPCSs gene and the NtPCSt gene may consist of a nucleotide sequence of SEQ ID NO: 5 or SEQ ID NO: 6.

In general, the guide sequence is any polynucleotide sequence having complementarity with a target polynucleotide sequence, which is sufficient to hybridize with the target sequence and to induce sequence-specific binding of a CRISPR complex to the target sequence. In addition, any nucleotide sequence that may be used for gene manipulation to reduce the expression or activity of the PCS gene or the protein expressed by the PCS gene may be used as the guide RNA without limitation. For example, the nucleotide sequence is a sequence capable of hybridizing with the PCS gene. In addition, a portion of the guide RNA nucleotide sequence may be modified to modify/enhance the function of the guide RNA. Further, in an embodiment, the degree of complementarity between a guide sequence and its corresponding target sequence, when optimally aligned using an appropriate alignment algorithm, is about 50%, about 60%, about 75%, about 80%, about 85%, about 90%, about 95%, about 97.5%, about 99% or more. Optimal alignment may be determined by using any suitable algorithm for aligning the sequences, and non-limiting examples thereof include a Smith-Waterman algorithm, a Needleman-Wunsch algorithm, a Burrows-Wheeler Transform-based algorithm (e.g., Burrows Wheeler Aligner), ClustalW, Clustal X, BLAT, Novoalign (Novocraft Technologies), ELAND (Illumine, San Diego, CA, USA), SOAP (available at soap.genomics.org.cn) and Ma (available at maq.sourceforge.net).

In an embodiment, the guide sequence is, for example, about 5 nucleotides, about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 35 nucleotides, about 40 nucleotides, about 45 nucleotides, about 50 nucleotides, about 75 or more nucleotides in length. In an embodiment, the guide sequence may be about 75 nucleotides, about 50 nucleotides, about 45 nucleotides, about 40 nucleotides, about 35 nucleotides, about 30 nucleotides, about 25 nucleotides, about 20 nucleotides, about 15 nucleotides, or about 12 nucleotides or less in length. The ability of the guide sequence to direct sequence-specific binding of the CRISPR complex to the target sequence may be assessed by any suitable assay. For example, components of the CRISPR system sufficient to form the CRISPR complex, including the guide sequence to be tested, may be provided to a host cell having the corresponding target sequence, such as by transfection with vectors encoding the components of the CRISPR sequence, for example, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and may be easily used by those skilled in the art.

In one specific embodiment, the sgRNA may bind to the PCS gene of at least one allele in the plant cell, and specifically, may bind to the PCS gene of all alleles. When the guide RNA binds to the PCS gene of all alleles to knock out the PCS gene, heavy metal absorption and transport may be inhibited not only in plant cells of the same generation but also in plant cells of subsequent generations.

In one specific embodiment, the target sequence used to knock out the PCS gene in the plant cell may be, for example, at least one site of Exon 1 and Exon 2 of the PCS gene. In addition, in one specific embodiment, it was confirmed that sgRNA produced to target Exon 2 of the PCS gene in the plant cell may induce the most frequent mutations.

In one specific embodiment, the plant cell is a plant cell in which a specific target site of the PCS gene is cleaved by sgRNA and the gene is knocked out. By the knockout, heavy metal absorption and transport of the plant cell may be suppressed.

The gene knockout may refer to regulation of gene activity, for example, inactivation by deletion, substitution of all or part of the gene (e.g., one or more nucleotides), and/or insertion of one or more nucleotides. The gene inactivation refers to a modification whereby gene expression is suppressed or gene expression is reduced (downregulation), or a protein with loss of its original function is encoded. In addition, gene regulation refers to a functional change in the gene, resulting from a structural modification of a protein, obtained by deletion of exon sites due to simultaneous targeting of both intron sites surrounding one or more exons of the target gene, expression of a dominant negative protein, and expression of a competitive inhibitor secreted in a soluble form, etc.

As used herein, the term “vector” refers to a means for expressing a target gene in a host cell. The vector includes, for example, viral vectors such as plasmid vectors, cosmid vectors, bacteriophage vectors, adenovirus vectors, retrovirus vectors, and adeno-associated virus vectors. The vector that may be used as the recombinant vector may be prepared by manipulating a plasmid often used in the art (e.g., V1k_GE, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phage or virus (e.g., SV40, etc.).

In the vector, a gene encoding a guide RNA binding to the PCS gene, a gene encoding the Cas9 protein, and a gene encoding the NLS may be operably linked to a promoter. The term “operably linked” refers to a functional linkage between a nucleotide expression control sequence (e.g., a promoter sequence) and another nucleotide sequence. The control sequence may be “operably linked” to regulate the transcription and/or translation of another nucleotide sequences.

In one specific embodiment, when the vector has a polynucleotide (P_U6) consisting of a nucleotide sequence of SEQ ID NO: 43 bound to the 5′-end of the gene encoding the guide RNA binding to the PCS gene, and five consecutive thymines at the 3′-end thereof, unstable guide RNA may be stabilized.

The vector, typically, may be constructed as a vector for cloning or a vector for expression. The vector for expression may be a vector commonly used to express foreign proteins in plants, animals or microorganisms in the art. The vector may be constructed through various methods known in the art.

The vector may be constructed using a prokaryotic cell or a eukaryotic cell as a host. For example, when the vector to be used is an expression vector and a prokaryotic cell is used as a host, the vector may generally include a strong promoter capable of driving transcription (e.g., CMV promoter, trp promoter, lac promoter, tac promoter, T7 promoter, etc.), a ribosome binding site for initiation of translation, and a transcription/translation termination sequence. When a eukaryotic cell is used as a host, an origin of replication operating in the eukaryotic cell, which is included in the vector, includes f1 origin of replication, SV40 origin of replication, pMB1 origin of replication, adeno origin of replication, AAV origin of replication, BBV origin of replication, etc., but is not limited thereto. In addition, a promoter derived from the genome of a mammalian cell (e.g., metallothionein promoter) or a promoter derived from a mammalian virus (e.g., an adenovirus late promoter, a vaccinia virus 7.5K promoter, an SV40 promoter, a cytomegalovirus promoter and a tk promoter of HSV) may be used, and it generally has a polyadenylation sequence as a transcription termination sequence.

As used herein, the term “CRISPR/Cas9 system” may include single guide RNA (sgRNA) including CRISPR RNA (crRNA) and transactivating crRNA (tracrRNA). Further, the CRISPR/Cas9 system may include a CRISPR associated protein 9 (Cas9) protein or a gene encoding the Cas9 protein and a nuclear localization signal (NLS) protein or a gene encoding the NLS protein.

In general, a widely known gene editing means, “Clustered Regularly Interspaced Short Palindromic Repeats (CRISPR) system”, collectively refers to transcripts and other elements involved in the expression of or directing the activity of CRISPR-associated (Cas) genes, including a Cas gene-encoding sequence, a trans-activating CRISPR (tracr) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a guide RNA or a CRISPR locus. In some embodiments, one or more elements of the CRISPR system are derived from a type I, type II, or type III CRISPR system. In some embodiments, one or more elements of the CRISPR system are derived from a particular organism including the endogenous CRISPR system, e.g., Streptococcus pyogenes. In general, the CRISPR system is characterized by elements that promote the formation of the CRISPR complex at the site of a target sequence (also referred to as a protospacer in the context of an endogenous CRISPR system). In the context of formation of the CRISPR complex, “target sequence” or “target gene” refers to a sequence to which a guide sequence is designed to have complementarity, where hybridization between a target sequence and a guide sequence promotes formation of the CRISPR complex. Full complementarity is not necessarily required, provided there is sufficient complementarity to cause hybridization and to promote formation of the CRISPR complex. The target sequence may include any polynucleotide, for example, DNA or RNA polynucleotides. In some embodiments, the target sequence is located in the nucleus or cytoplasm of a cell. In some embodiments, the target sequence may exist within an organelle of a eukaryotic cell, for example, mitochondrion or chloroplast.

When the Cas protein forms a complex with two RNAs called CRISPR RNA (crRNA) and trans-activating crRNA (tracrRNA), an active endonuclease or nickase is formed. Non-limiting examples of the Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, an amino acid sequence of Streptococcus pyogenes Cas9 protein may be found in the SwissProt database under accession number of Q99ZW2. In some embodiments, the unmodified CRISPR enzyme, for example, Cas9 has DNA cleavage activity.

In some embodiments, the CRISPR enzyme is a Cas9 protein, and the Cas9 protein may be at least one Cas9 protein selected from the group consisting of a Cas9 protein derived from Streptococcus pyogenes, a Cas9 protein derived from Campylobacter jejuni, a Cas9 protein derived from Streptococcus thermophiles, a Cas9 protein derived from Streptocuccus aureus, and a Cas9 protein derived from Neisseria meningitidis, and specifically, a Cas9 protein derived from Streptococcus pyogenes. In some embodiments, the Cas9 protein is codon-optimized for expression in eukaryotic cells, and when the Cas9 protein derived from Streptococcus pyogenes is used, the expression or activity of the PCS gene or the protein expressed by the PCS gene may be reduced to the maximum.

In some embodiments, the Cas9 protein may include a nuclear localization sequence or signal (NLS) at the 5′- or 3′-end, or both ends of the Cas9 protein to be located in the nucleus of a eukaryotic cell, wherein the NLS may be one or more.

As used herein, the term “NLS” refers to an amino acid sequence that serves to transport a specific substance (e.g., protein) into the nucleus of a cell, and generally acts to transport the substance into the nucleus of the cell via nuclear pores. The NLS is not required for the activity of CRISPR complex in eukaryotes, but it is believed that this sequence is included to enhance the activity of the system, particularly, to target a nucleic acid molecule in the nucleus.

In addition, RNA genetic scissors (RNA-guided clustered regularly interspaced short palindrome repeats (CRISPR)-associated nuclease Cas9) provides a breakthrough technology for target gene knockout, transcription activation, and inhibition using single guide RNA (sgRNA) (i.e., crRNA-tracrRNA fusion transcript), and this technology is known to target numerous gene loci.

Cas9 (or Cpf1) protein refers to an essential protein element in the CRISPR/Cas9 system, and information on the Cas9 (or Cpf1) gene and protein may be obtained from GenBank of the National Center for Biotechnology Information (NCBI), but is not limited thereto. With regard to the CRISPR-associated gene encoding Cas (or Cpf1) protein, about 40 or more different Cas (or Cpf1) protein families are known to exist. 8 CRISPR subtypes (Ecoli, Ypest, Nmeni, Dvulg, Tneap, Hmari, Apern, and Mtube) may be defined according to a specific combination of the cas gene and repeat structure. Therefore, each of the CRISPR subtypes may form a repeating unit to form a polyribonucleotide-protein complex.

When DNA encoding the Cas9 is delivered to an individual or cell, the DNA may generally (but not necessarily) include a regulatory element (e.g., a promoter) operable in a target cell. The promoter for Cas9 expression may be, for example, a CMV, EF-I a, EFS, MSCV, PGK, or CAG promoter. A promoter for gRNA expression may be, for example, a HI, EF-Ia, tRNA or U6 promoter. The promoter may be tissue-specific or cell-specific.

Still another aspect provides a method of preparing a plant having reduced heavy metal absorption, the method including transforming a plant cell with a CRISPR-Cas9 recombinant vector including a single guide RNA targeting the PCS gene. Detailed descriptions of the PCS gene, single guide RNA, CRISPR-Cas9 system, vector, plant cell, etc. are as described above.

In the transforming, the method is not particularly limited, as long as it is a transformation method well-known in the art and capable of transforming a tobacco gene. For example, the transforming may be performed by one method selected from the group consisting of an Agrobacterium-mediated transformation method, a polyethylene glycol (PEG)-mediated protoplast transformation method, a gene gun method, an electrode transformation method, a vacuum infiltration transformation method, and a silicon carbide fiber-mediated transformation method.

In one specific embodiment, considering characteristics and a transformation rate of tobacco, the recombinant vector including the single guide RNA targeting the PCS gene may be transformed into a plant cell by the Agrobacterium-mediated transformation method.

### Advantageous Effects of Disclosure

In a plant cell according to an aspect, in which expression or activity of a phytochelatin synthase (PCS) gene or a protein encoded by the PCS gene is reduced, pathways involved in absorption, storage, or transport of heavy metals by binding to heavy metal ions in the plant cells are inhibited, and thus there is an effect of reducing the content or absorption of heavy metals in the plant cell or a plant including the plant cell.

A plant cell according to another aspect, in which expression or activity of a PCS gene or a protein encoded by the PCS gene is reduced, may overcome a growth inhibition phenomenon, which is the limit of existing plants with reduced heavy metals.

### BRIEF DESCRIPTION OF DRAWINGS

FIGS. 1A and 1B show alignment of a part of known reference nucleotide sequences and part of a nucleotide sequence of an NtHMA gene of KB108, specifically, FIG. 1A shows a result of comparing exon 2 sites in NtHMAα genes (gRNA_A4_e2: a site specific to the NtHMAα gene, designed to bind with sgRNA) and FIG. 1B shows a result of comparing exon 2 sites in the NtHMAβ genes (gRNA_B4_e2: a site specific to the NtHMAβ gene, designed to bind with sgRNA);

FIG. 2 shows a structure of a genetic scissors expression vector including two sgRNAs;

FIGS. 3A to 3D show photographs of each stage of plant tissue culture after transformation by an Agrobacterium-mediated transformation method, specifically, FIG. 3A shows a stage of cutting leaf tissues and transforming them by culturing with Agrobacterium, FIG. 3B shows a stage of inducing callus differentiation and shoot differentiation, FIG. 3C shows a stage of inducing root differentiation, and FIG. 3D shows a state of a plantlet in which differentiation is completed;

FIG. 4 shows results of electrophoresis by amplifying each target gene site with respect to gDNA of 24 individuals of tissue cultures to examine the occurrence and patterns of mutations (Lane M: 1 kb DNA marker, Lanes 1 to 7: amplification results of NtHMAα site, Lanes 9 to 15: amplification results of NtHMAβ site, Lanes 17 to 21: amplification results of NtPCSs site, Lanes 23 to 27: amplification results of NtPCSt site, Lanes 8, 16, 22, 28: Non-template control (NTC));

FIGS. 5A and 5B show results of sequencing analysis of NtHMAβ sites of a wild-type KB108 and a variant, specifically, FIG. 5A shows a result of sequencing analysis of the KB108 and FIG. 5B shows a result of sequencing analysis of the variant, in which a site into which an adenine base is inserted, compared to the nucleotide sequence of the wild-type, is indicated by a red arrow;

FIG. 6 shows results of electrophoresis after performing a gene amplification reaction to examine whether a gene introduced into an F₁ plant is inherited;

FIG. 7 shows a graph showing relative values of the cadmium contents, based on the cadmium content of a control group, by summarizing results of primary and secondary cadmium content analysis performed in a hydroponic culture system;

FIG. 8 shows results of analyzing the cadmium contents of plants cultured in a greenhouse soil environment;

FIG. 9 shows a graph of comparing growth characteristics of an NtHMAαβ variant and an NtPCSst variant, as compared with the control KB108 (Height: height of plant, #of leaves: number of leaves, Weight: weight of leaves); and

FIG. 10 shows photographs in which the growth status of the NtHMAαβ variant and the NtPCSst variant, as compared with the control KB108, was observed with the naked eye.

### MODE OF DISCLOSURE

Hereinafter, the present disclosure will be described in more detail with reference to exemplary embodiments. However, these exemplary embodiments are only for illustrating, and the scope of the present disclosure is not limited to these exemplary embodiments.

### Example 1. Production of Tobacco with Reduced Cadmium Using NtPCS Gene-Targeting CRISPR/Cas9 System

(1) Construction of Gene Vehicle

(1.1) Examination of Nucleotide Sequences of Heavy Metal-Related Genes (NtHMA and NtPCS)

Two types of genes related to cadmium absorption and transport in tobacco were selected; NtHMAα and NtHMAβ, which are genes encoding proteins acting as gates that open and close when transporting divalent metal ions absorbed into roots to other tissues; and NtPCS, which is a gene encoding a protein involved in the storage and transport of metal ions by binding to metal ions in cells.

To examine nucleotide sequences of the NtHMA and NtPCS genes in a burley tobacco variety (KB108) which is a research target plant, primers specific to each gene were prepared, based on the nucleotide sequence information published in the National Center for Biotechnology Information (NCBI) database, and a gene amplification reaction (PCR: Polymerase Chain Reaction) was performed. The sequences of the primers specific to each gene and PCR conditions are shown in Table 1 below.

TABLE 1

| | | Amplifi- | | | | |
|---|---|---|---|---|---|---|
| | | cation | PCR conditions | | | SEQ |
| | Nucleotide | size | Annealing | Extension | | ID |
| Primer name | sequence (5′→3′) | (bp) | Tmp | time | Cycles | NO: |
| F_HMAα | GAAACAAAGAAG | 2937 | 62° C. | 90 sec | 35 | 7 |
| | TTGAGCAAGAGC | | | | | |
| | TATT | | | | | |
| R HMAα | AGCCTTAGTGAG | | | | | 8 |
| | ATGATTTATAACA | | | | | |
| | CAA | | | | | |
| F_HMAß | GACACAAAGAAT | 2374 | 62° C. | 90 sec | 35 | 9 |
| | CTGAGCAAGAGC | | | | | |
| | TATT | | | | | |
| R_HMAß | AGCTAGAGTAGG | | | | | 10 |
| | ACCACACATTAAT | | | | | |
| | TCT | | | | | |
| F_PCSs | AAATGGCGATGG | 927 | 68° C. | 90 sec | 35 | 11 |
| | CGGGTTTGTAT | | | | | |
| R_PCSs | GTCGGGAAGGAT | | | | | 12 |
| | TAGAACACAAATT | | | | | |
| | CAC | | | | | |
| F_PCSt | AATGGCGATGGC | 2731 | 68° C. | 90 sec | 35 | 13 |
| | GGGTTTATATC | | | | | |
| R_PCSt | AGTCCGGAAGGA | | | | | 14 |
| | TAGGAACACAGA | | | | | |
| | TT | | | | | |

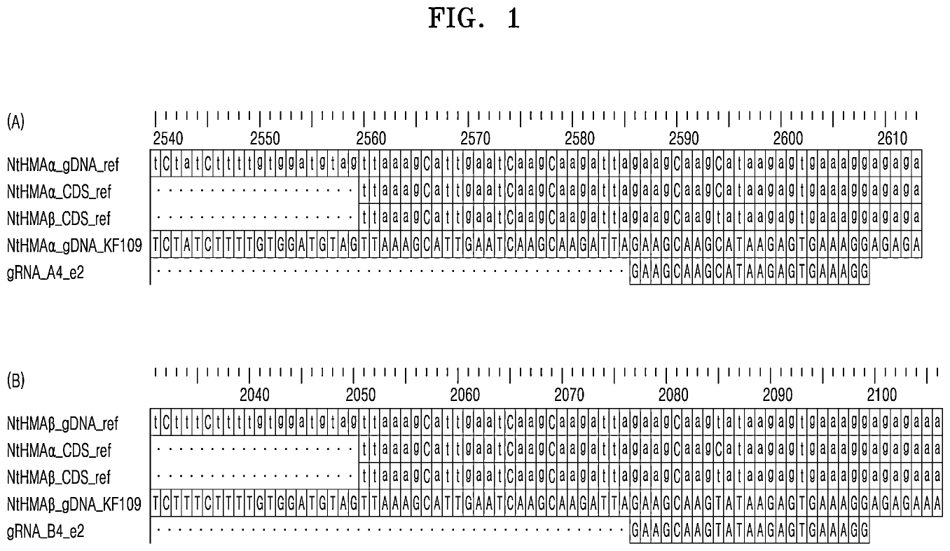

As shown in FIG. 1, as a result of analyzing the nucleotide sequence by amplifying the gDNA region of the KB108 variety, the nucleotide sequence was found to mostly correspond to the published nucleotide sequence. Specifically, as a result of aligning and comparing a part of the published reference nucleotide sequences (Database accession Nos. HF675180.1 and HF937054.1) with the nucleotide sequences of NtHMA genes of KB108, when comparing each exon 2 site in the NtHMAα gene (A) and NtHMAβ gene (B), most of them was confirmed to correspond to each other, and the site was designed such that sgRNA specifically bind to each gene.

The results of examining homology between the NtPCSs gene (only some sequences) of the KB108 variety, a reference genome nucleotide sequence of N. tabacum (TN90), and a reference genome nucleotide sequence of N. sylvestris are shown in Table 2 below, and the results of examining homology between the NtPCSt gene of the KB108 variety, a reference genome nucleotide sequence of N. tabacum (TN90), and a reference genome nucleotide sequence of N. tometosiformis are shown in Table 3 below.

TABLE 2

| Homology comparison of | PCSs_KB108 | PCSs_TN90 | PCSs_Nsyl |
|---|---|---|---|
| PCSs gene | (KT&G) | (NW 015845165.1) | (NW 009518934.1) |
| PCSs_KB108(KT&G) | ID | 1.000 | 1.000 |
| PCSs_TN90(NW_015845165.1) | 1.000 | ID | 1.000 |
| PCSs Nsyl(NW 009518934.1) | 1.000 | 1.000 | ID |

TABLE 3

| Homology comparison of | PCSt_KB108 | PCSt_TN90 | PCSt_Ntom |
|---|---|---|---|
| PCSt gene | (KT&G) | (NW 015825368.1) | (NW 008939610.1) |
| PCSt_KB108(KT&G) | ID | 1.000 | 0.986 |
| PCSt_TN90(NW_015825368.1) | 1.000 | ID | 0.986 |
| PCSt Ntom(NW 008939610.1) | 0.986 | 0.986 | ID |

The results of comparing nucleotide sequences of the NtPCSs gene derived from Nicotiana sylvestris and the NtPCSt gene derived from Nicotiana tomentosiformis with the reference genome nucleotide sequence of N. tabacum (TN90) showed 100% homology, as confirmed in Tables 2 and 3. Further, the NtPCSs gene (only some sequences) showed 100% homology to the reference genome data of N. sylvestris, and the NtPCSt gene showed about 98.6% homology to the reference genome data of N. tometosiformis.

(1.2) Design of Genetic Scissors Block and Recombination of Vehicle

The nucleotide sequences of the NtHMAα, NtHMAβ, NtPCSs and NtPCSt genes obtained in Example 1-(1) were compared, and a part specific to each gene was selected as a genetic scissors guide (sgRNA). A total of 5 types of recombinant vehicles were completed by cloning gene scissors expression blocks selected as gene vehicles expressed in plants.

Information about the gene vehicles including each of six sgRNAs capable of specifically binding to the NtHMAα and NtHMAβ genes is shown in Table 4 below.

TABLE 4

| | | | | | SEQ ID |
|---|---|---|---|---|---|
| Vector | sgRNA | Target | Site | Sequence | NO: |
| Vlk_HMAA6 | gRNA_A1 | HMAα | Exon 1 | TCTTTCTTACCAATT | 15 |
| | | | | TGTTG | |
| | gRNA_A2 | | Intron 1 | TGTTTGTACAAGCTT | 16 |
| | | | | TTAGA | |
| | gRNA_A3 | | Intron 1 | ATGGTAACTTCAATA | 17 |
| | | | | ATTATA | |
| | gRNA_A4 | | Exon 2 | AAGCAAGCATAAGA | 18 |
| | | | | GTGAA | |
| | gRNA_A5 | | Exon 2 | CCACACCTCTAAAAA | 19 |
| | | | | TAAT | |
| | gRNA_A6 | | Intron 2 | TCATATAAATTGGGA | 20 |
| | | | | CAAA | |
| Vlk_HMAB6 | gRNA_B1 | HMAß | Exon 1 | CAATTTGTTGCTGAG | 21 |
| | | | | AAATG | |
| | gRNA_B2 | | Intron 1 | AGTGGAGAAAAGAT | 22 |
| | | | | GAAGAA | |
| | gRNA_B3 | | Intron 1 | ATGGTAACTACAATA | 23 |
| | | | | ATTATA | |
| | gRNA_B4 | | Exon 2 | AAGCAAGTATAAGA | 24 |
| | | | | GTGAA | |
| | gRNA_B5 | | Exon 2 | GATTCCTCCAATTAT | 25 |
| | | | | TTTT | |
| | gRNA_B6 | | Exon 2 | CCACACCCCTAAAAA | 26 |
| | | | | TAAT | |

Information about the gene vehicles including each of two sgRNAs capable of specifically binding to the NtPCSs and NtPCSt genes is shown in Table 5 below.

TABLE 5

| | | | | | SEQ ID |
|---|---|---|---|---|---|
| Vector | sgRNA | Target | Site | Sequence | NO: |
| Vlk_PCS_S | gRNA_S1 | PCSs | Exon 1 | AAGCGAAATCAACA | 1 |
| | | | | GCCGGAG | |
| | gRNA_S2 | | Exon 2 | GGCATTCAAGACCA | 2 |
| | | | | TGGAA | |
| Vlk_PCS_T | gRNA_T1 | PCSt | Exon 1 | CGAGTTCTTCCGTC | 3 |
| | | | | GCCTC | |
| | gRNA_T2 | | Exon 2 | GGCATTCAAGACCA | 4 |
| | | | | TAGAA | |
| Vlk_PCS_ST | gRNA_ST1 | PCSs | Exon 1 | TAGAAGCGAAATCA | 5 |
| | | PCSt | | ACAGC | |
| | gRNA_ST2 | | Exon 2 | GCCATCCAGAATGG | 6 |
| | | | | AACAA | |

In detail, pBI121, which is a binary vector replicable in E. coli and agrobacterium and is a vector widely used for plant transformation, was digested with HindIII and EcoRI and prepared to clone a GE_block which is needed for the CRISPR/Cas9 system. The GE_block consists of, in this order, a CaMV 35S promoter with dual enhancer (P_35Sd), a multi cloning site (MCS) for cloning Cas9 block, a CaMV 35S terminator (T_35S), a linker sequence, and a multi cloning site (MCS) for cloning sgRNA block. HindIII and EcoRI recognition nucleotide sequences were added at both ends thereof. Each block of the GE_block was prepared by DNA synthesis, and sequentially cloned to complete the GE_block. A recombinant vehicle (V1k_GE) was prepared by ligation of pBI121 and GE_block, each digested with HindIII and EcoRI. CRISPR/Cas9_block consists of a block (Cas9_block) consisting of a Cas9 coding sequence (CDS) and a C-terminus nuclear localization sequence (NLS), and BamHI and SacI recognition nucleotide sequences at both ends thereof; and a block capable of expressing sgRNA (sgRNA_PMT) consisting of a U6 promoter (P_U6, SEQ ID NO: 43), sgRNA, and poly T. Two types of sgRNA blocks capable of specifically bind to NtPCS genes (NtPCSs and/or NtPCSt gene) were linked into one contiguous DNA through an overlap extension PCR technique to complete sgRNA_PMT. SalI and SpeI recognition nucleotide sequences exist at both ends of sgRNA_PMT.

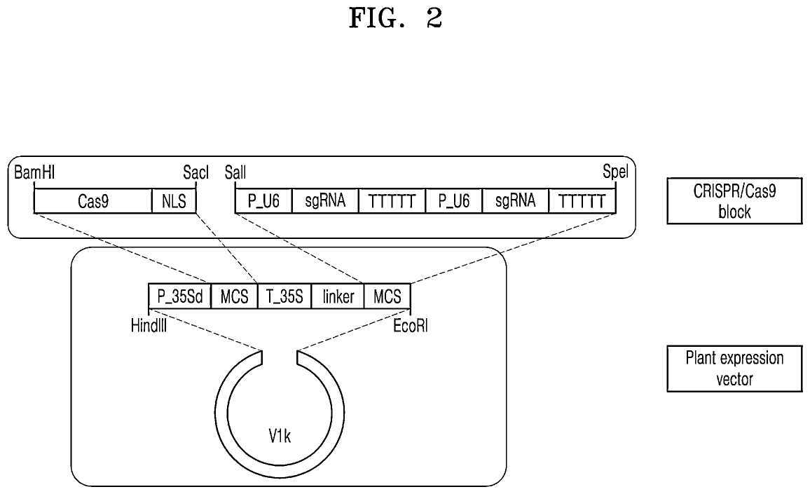

As shown in FIG. 2, V1k_GE and CRISPR/Cas9_block, each digested with BamHI and SacI, were ligated together through a ligation reaction, and then digested with SalI and SpeI, and sgRNA_PMT was inserted to construct a gene vehicle.

(2) Introduction of Recombinant Vehicle into Gene Transfer Microorganism (Agrobacterium)

A plant vehicle was transformed into Agrobacterium LBA4404 strain by a freeze-thaw method.

In detail, the Agrobacterium strain was inoculated in a YEP liquid medium (10 g of yeast extract, 10 g of Bacto™ peptone, 5 g of NaCl), and then incubated with shaking under conditions of 28° C. and 250 rpm for 16 hours. Cells were separated by centrifuging the culture medium under conditions of a speed of 3,000 g and 4° C. for 20 minutes, and suspended in 20 mM CaCl₂) to prepare competent cells. 5 μL of plasmid DNA (vehicle for plants) was added to 100 μL of competent cells, and incubated in liquid nitrogen for 5 minutes and at 37° C. for 5 minutes. 1 mL of YEP liquid medium was added and incubated with shaking under conditions of 28° C. and 250 rpm for 2 hours. 100 μL of the culture medium was spread on a YEP solid medium containing 100 mg/L of kanamycin, and then incubated at 28° C. for 3 days. Respective single colonies were sub-cultured, and whether the plasmid DNA was transformed was confirmed by PCR.

(3) Plant Tissue Culture

(3.1) Plant Transformation

The Agrobacterium strain, in which transformation of the gene vehicle was confirmed in Example 1-(2) above, was cultured in a YEP liquid medium (including 70 mg/L kanamycin and 70 mg/L streptomycin) under conditions of 28° C. for 24 hours. In addition, the leaves of the plant at 1 month after germination were sterilized with 70% ethanol and Clorox, and cut into 3 mm×3 mm slices, which were placed in a Petri dish containing 5 ml of MS liquid medium, and then 1 mL of the culture solution of Agrobacterium strain was evenly sprayed to prepare Tobacco leaf slices. The Tobacco leaf slices were then incubated at 25° C. in dark conditions for 48 hours.

(3.2) Plant Tissue Culture

The leaf slices were washed with sterile distilled water (including 200 ug/ml cefotaxim) four times, and then explanted in a shooting medium (MS medium, 2 mg/L BA, 0.1 mg/L NAA, 200 mg/L cefotaxim, 100 mg/L kanamycin), and incubated under conditions of 25° C., 16 h/8 h photoperiod, and sub-cultured with a fresh medium every 2 weeks to perform washing and explanting on the selection medium.

In addition, shoots differentiated from the leaf slices were cut and explanted in a rooting medium (MS medium, including 200 mg/L cefotaxime), and incubated under conditions of 25° C. and 16 h/8 h photoperiod to perform explanting on the rooting medium.

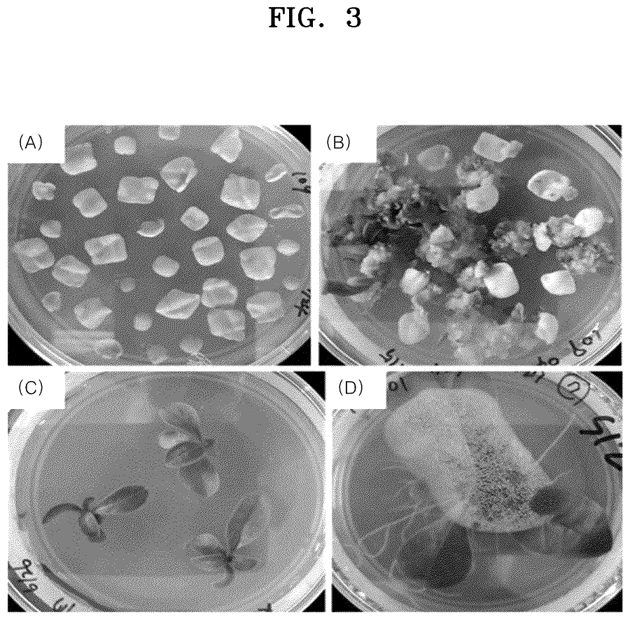

As a result, as shown in FIG. 3, after transformation into tobacco leaf tissues by the Agrobacterium-mediated transformation method, whether callus differentiation, leaf differentiation, and root differentiation occurred well in sequence was confirmed. Through tissue culture, 102 individuals of tissue culture plantlets with leaves, stems, and roots were obtained.

(4) Selection of Variant

(4.1) Examination of Mutation Occurrence and Patterns Thereof in Target Gene

Each part of the leaves of the tissue cultures obtained in Example 1-(3) was collected, and gDNA was extracted therefrom, and then each target gene site was amplified through a PCR reaction. The nucleotide sequence of the amplified gene product was analyzed, and 50 tissue cultures with mutations in the NtHMA gene and 60 tissue cultures with mutations in the NtPCS gene were selected.

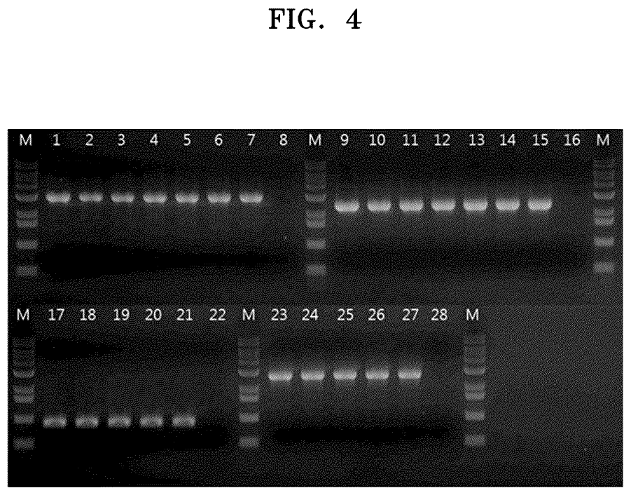

In detail, 100 mg of healthy leaf tissue was sampled and uniformly ground, and gDNA was extracted and purified using a commercial kit (e.g., Nucleospin 96 plant II, Macherey Nagel, Germany) using a silica column. FIG. 4 shows results of electrophoresis by amplifying each target gene site with respect to gDNAs of 24 individuals of tissue cultures to examine the occurrence and patterns of mutations.

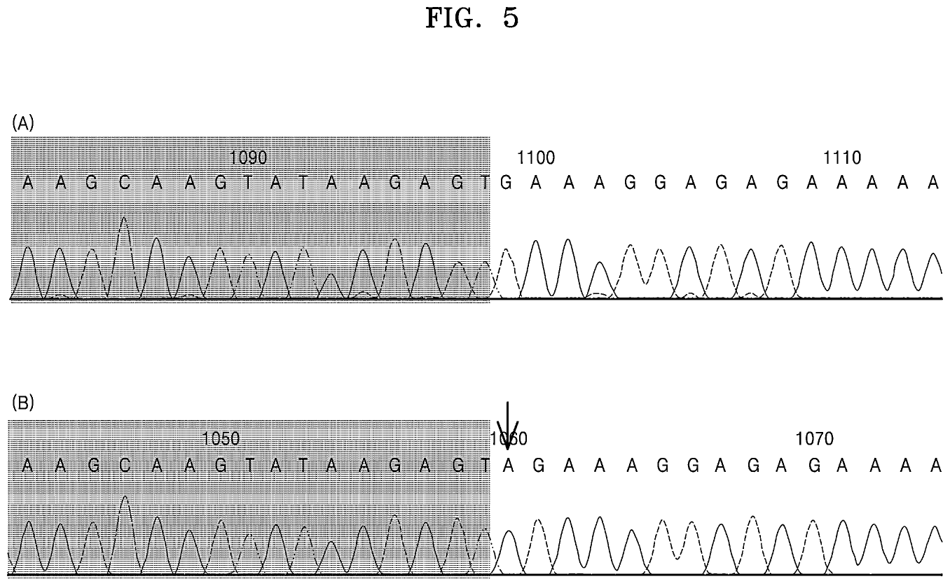

FIG. 5 shows an illustration of base sequence analysis by amplifying the target gene site through PCR, after extraction/purification of gDNA from the leaf tissue. As shown in FIG. 5, as a result of nucleotide sequence analysis of the NtHMAβ sites of the wild-type KB108 and the variant, it was confirmed that an adenine base was inserted in the nucleotide sequence of the variant, as compared with the nucleotide sequence of the wild-type.

The mutation patterns of the NtHMA gene were classified according to the site, and as a result, as shown in Table 6 below, mutation rates were 44% to 48% at the sites of gRNA_HMA_A4 and gRNA_HMA_B4 prepared by targeting exon 2, and it was confirmed that the mutation occurred most frequently at the exon 2 site of the NtHMA gene.

TABLE 6

| | | | | Number of | |
|---|---|---|---|---|---|
| | | | Number of | tissue | Mutation |
| | Target | Site | mutation | culture | rate (%) |
| gRNA_HMA_A1 | HMA | Exon 1 | 1 | 25 | 4 |
| | α | | | | |
| gRNA_HMA_A2 | | Intron 1 | 3 | | 12 |
| gRNA_HMA_A3 | | Intron 1 | 0 | | 0 |
| gRNA_HMA_A4 | | Exon 2 | 12 | | 48 |
| gRNA_HMA_A5 | | Exon 2 | 2 | | 8 |
| gRNA_HMA_A6 | | Intron 2 | 10 | | 40 |
| gRNA_HMA_B1 | HMA | Exon 1 | 5 | 25 | 20 |
| | β | | | | |
| gRNA_HMA_B2 | | Intron 1 | 7 | | 28 |
| gRNA_HMA_B3 | | Intron 1 | 0 | | 0 |
| gRNA_HMA_B4 | | Exon 2 | 11 | | 44 |
| gRNA_HMA_B5 | | Exon 2 | 5 | | 20 |
| gRNA_HMA_B6 | | Exon 2 | 2 | | 8 |

The mutation patterns of the NtPCS gene were classified according to the site, and as a result, as shown in Table 7 below, mutation rates were 45% to 70% at the sites of gRNA_PCSs_e2, gRNA_PCSt_e2, and gRNA_PCSst_e2 prepared by targeting exon 2, and it was confirmed that the mutation occurred most frequently at the exon 2 site of the NtPCS gene.

TABLE 7

| | | | | Number of | |
|---|---|---|---|---|---|
| | | | Number of | tissue | Mutation |
| | Target | Site | mutation | culture | rate (%) |
| gRNA_PCSs_e1 | PCSs | Exon 1 | 0 | 20 | 0 |
| gRNA_PCSs_e2 | | Exon 2 | 12 | | 60 |
| gRNA_PCSt_e1 | PCSt | Exon 1 | 0 | 20 | 0 |
| gRNA_PCSt_e2 | | Exon 2 | 14 | | 70 |
| gRNA_PCSst_e1 | PCSs | Exon 1 | 0 | 20 | 0 |
| gRNA_PCSst_e2 | PCSt | Exon 2 | 9 | | 45 |

(4.2) Obtaining of Seeds of F1 Generation and Selecting of Introduced Gene-Removed Plants

50 individuals of the NtHMA gene variant and 60 individuals of the NtPCS gene variant, in which the mutations were identified, were transplanted into pots filled with bed soil, and grown in a greenhouse. Seeds of F₁ generation were obtained through self-pollination to remove the gene block introduced for CRISPR/Cas9 expression. The seeds of F₁ generation were seeded in a 128-hole tray and grown for 30 days, then the leaves were collected and uniformly grounded, and then gDNA was extracted and purified using a silica column and a commercial kit (e.g., Nucleospin 96 plant II, Macherey Nagel, Germany). PCR was performed using a 35S promoter and primers specifically amplifying the Cas9 DNA block. Sequences of the primers are shown in Table 8 below.

TABLE 8

| | | PCR | | SEQ ID |
|---|---|---|---|---|
| Primer | Sequence | length | Note | NO: |
| F_C9 | GACCATCCTGGACTTCCT | 420 bp | Cas 9 | 27 |
| | GAAGAGC | | detected | |
| R_C9 | TGCAGGTAGTACAGGTAC | | | 28 |
| | AGCTTCTCG | | | |
| F_35S | GCTCCTACAAATGCCATC | 195 bp | 35S promoter | 29 |
| | A | | detected | |
| R_35S | GATAGTGGGATTGTGCGT | | | |
| | CA | | | |

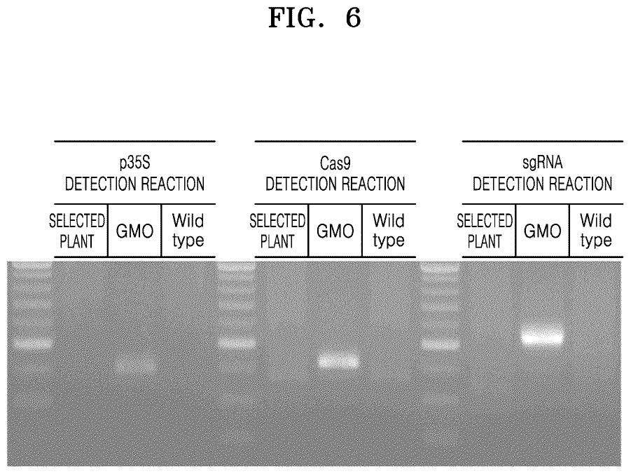

As a result, as shown in FIG. 6, plants in which the 535S promoter and the Cas9 DNA block were not detected, were selected.

(4.3) Genotyping Information of Final Selected Plants

The final selected F1 plants (108_PMTm_F1) did not include the introduced gene, and were plants, in which homozygous mutation occurred in each of the NtHMA genes and NtPCS genes. The genotypes of the final plants, in which mutations were induced in each of the NtHMA genes and the NtPCS genes, are shown in Table 9 below.

TABLE 9

| | | | Mutation |
|---|---|---|---|
| | Target | Mutation site | pattern |
| A6_02 | HMAα | Exon 2 | GT del |
| B6_02 | HMAβ | Exon 2 | T ins |
| AB_01 | HMAα | Exon 2 | GTGA del |
| | HMAβ | Intron 1 | T del |
| | | Exon 2 | A ins |
| | | Exon 2 | T ins |
| T_05 | PCSt | Exon 2 | T ins |
| ST_01 | PCSs | Exon 2 | A ins |
| | PCSt | Exon 2 | A ins |

In addition, the gDNA nucleotide sequences, CDS nucleotide sequences, and amino acid sequences of the PCSs genes and PCst genes of the control plant (KB108), and the plant (PCSst), in which mutations were induced in the NtPCSs and NtPCSt genes are summarized and shown in Table 10 below.

TABLE 10

| Plant | Type | SEQ ID NO: | Abbreviation |
|---|---|---|---|
| KB108 | PCSs_gDNA | 31 | KB108_PCSs_gDNA |
| | PCSs_CDS | 32 | KB108 PCSs_CDS |
| | PCSs_amino acid | 33 | KB108 PCSs_AA |
| | PCSt_gDNA | 34 | KB108_PCSt_gDNA |
| | PCSt_CDS | 35 | KB108_PCSt_CDS |
| | PCSt_amino acid | 36 | KB108_PCSt_AA |
| PCSst | PCSs_gDNA | 37 | PCSst PCSs_gDNA |
| | PCSs CDS | 38 | PCSst PCSs_CDS |
| | PCSs_amino acid | 39 | PCSst_PCSs_AA |
| | PCSt_gDNA | 40 | PCSst_PCSt_gDNA |
| | PCSt_CDS | 41 | PCSst_PCSt_CDS |
| | PCSt_amino acid | 42 | PCSst_PCSt_AA |

(In the amino acid sequence, * represents a stop codon, and the stop codon in the middle of the amino acid sequence represents an early stop codon caused by a mutation in the nucleic acid sequence.)

For reference, amino acid sequences of SEQ ID NO: 39 and SEQ ID NO: 42 are as follows.

SEQ ID NO: 39:
MAMAGLYRRV LPSPPAVDFA STEGKQLFLE AIQNGNNGRI
FQVDLLFSDT V*TGLLWFG* PFHGLECPCY *SRKKMER
SEQ ID NO: 42:
MAMAGLYRRV LPSPPAVDFA STEGKQLFLE AIQNGNNGRI
FQVDLLFSDT V*TGLLWFG* PFYGLECPCY *SRKKMERAL
EMV**IYVGL L*ASGEG*S* RDLFWESGMF GSLCRSEGRS
FSL*S*YY** LP*TSHGLHY ***LSSDLII S*RPF*ADRF
GPLFAYWWLS RGKGYGTDSR CCEV*ISSSL GSPPSPLGSH
EHN**SYRIT *GVYANY*AS QSSCTAIYPE L*T*ELGHYL
KAFDG*SSCP VKF*ECEGHK RCSLYCSFKS TFKFC*IHKV
DSGSSKARGE WSKFE*RGER KASYQGRGIE TSAGHSSL*A
CHKHFIFKKF YLPVKSSIRQ QFG*CCRKHL LPRSRSFCRK
IWFIG*VLLS PNMC*MLQSY RGQFCYSGVW DSCKWEWGAG
G*CSGPYISS KD*LLSLRAS WLLANAPCK* RCADSTIAGI
TSTYMVSNKR YEGLAGNREP CLSREPASFA ARRDFAPART
VPPPQEMQG* QGRRRFSCTS LL

### Example 2. Analysis of Cadmium Content in Tobacco Plant with Mutated NtPCS Gene

(1) Analysis of Cadmium Content Using Hydroponic Culture System

The germinated plants were transplanted to a hydroponic culture system, and then the flower stalk was cut from each plant grown for 80 days, and 2 weeks later, all leaves of each plant were harvested, and the harvested leaves were dried in a dry oven at 65° C. for 48 hours. Then, the leaves were put in a container containing glass beads and ground using a gyro-shaker. Experiments were conducted by dividing primary and secondary experiments according to the time of obtaining the plant variants. The cadmium contents of KB108 (wild-type, control) and variants were quantitatively analyzed by a GC/MS analytical technique. The results of the two experiments were expressed as a relative amount compared to the cadmium absorption amount of the control plant.

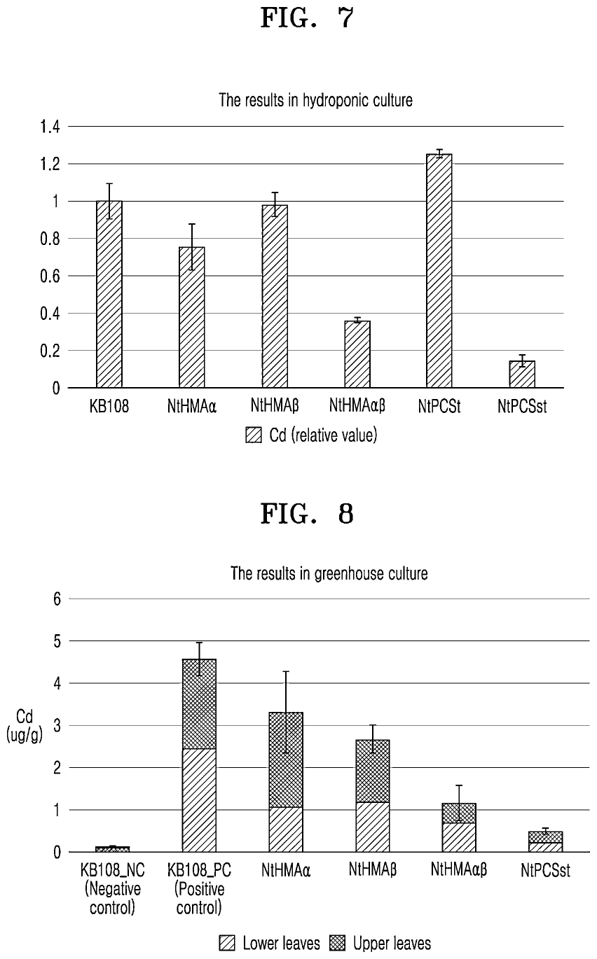

As a result, as shown in FIG. 7 and Table 11 below, the cadmium content in the leaves of the plants with the mutated NtHMAα or NtHMAβ gene was reduced by 25% or 2%, as compared with that of the control group, and the cadmium content in the leaves of the plants with both the mutated NtHMAα and NtHMAβ genes was reduced by 64%, as compared with that of the control group. In particular, the cadmium content in the leaves of the plants with both the mutated NtPCSs and NtPCSt genes was reduced by 85%, as compared with that of the control group, indicating a remarkable cadmium reduction effect. These plants show the excellent cadmium reduction effect, even as compared with the plants with both the mutated NtHMAα and NtHMAβ genes.

TABLE 11

| | Cadmium |
|---|---|
| Plant | (Relative value) |
| KB108 (Control) | 1.00 |
| NtHMAα | 0.75 |
| NtHMAβ | 0.98 |
| NtHMAαβ | 0.36 |
| NtPCSt | 1.25 |
| NtPCSst | 0.15 |

(2) Analysis of Cadmium Content in Greenhouse Environment

To examine whether the effect of reducing cadmium absorption of plants with the mutated NtPCS gene is maintained in the soil environment, a cadmium absorption experiment was performed in a greenhouse environment. In detail, each of the germinated plants was transplanted to a pot in a greenhouse environment, and then the flower stalk was cut from each plant grown for 60 days, and 2 weeks later, all leaves of each plant (lower leaves, upper leaves, or entire leaves) were harvested, and the harvested leaves were dried in a dry oven at 65° C. for 48 hours. Then, the leaves were put in a container containing glass beads and ground using a gyro-shaker. The cadmium contents of KB108 (wild-type, control) and variants were quantitatively analyzed by a GC/MS analytical technique.

As a result, as shown in FIG. 8 and Table 12 below, cadmium was rarely detected in a control group without cadmium treatment (KB108_NC), whereas a very high level of cadmium above the limit of quantitation (LOQ) of the assay was detected in a control group with cadmium treatment (KB108_PC). The cadmium content in the leaves of the plants with the mutated NtHMAα or NtHMAβ gene was reduced by about 26%, as compared with that of the control group, and the cadmium content in the leaves of the plants with both the mutated NtHMAα and NtHMAβ genes was reduced by about 77%, as compared with that of the control group. In particular, the cadmium content in the leaves of the plants with both the mutated NtPCSs and NtPCSt genes was reduced by about 87%, as compared with that of the control group, indicating a remarkable cadmium reduction effect. These plants show the excellent cadmium reduction effect, even as compared with the plants with both the mutated NtHMAα and NtHMAβ genes.

TABLE 12

| | Lower leaves | Upper leaves | Whole leaves |
|---|---|---|---|
| | Cadmium | Cadmium | Cadmium |
| | content | content | content |
| Plant | (μg/g) | (μg/g) | (μg/g) |
| KB108_NC | 0.08 | 0.04 | 0.13 |
| (Negative control) | | | |
| KB108_PC | 2.44 | 2.13 | 4.57 |
| (Positive control) | | | |
| NtHMAα | 1.05 | 2.27 | 3.31 |
| NtHMAβ | 1.19 | 1.48 | 2.67 |
| NtHMAαβ | 0.68 | 0.48 | 1.16 |
| NtPCSst | 0.23 | 0.26 | 0.49 |

Taken together, the cadmium content in the leaves of plants with both the mutated NtPCSs and NtPCSt genes was reduced by 86%, as compared with that of the control, under the hydroponic environment, and reduced by 87%, as compared with the control, under the soil environment, indicating the highest cadmium reduction effect.

### Example 3. Examination of Growth Characteristics of Tobacco Plant with Mutated NtPCS Gene

To examine whether the NtPCS mutant plants are able to overcome a growth inhibition phenomenon, which is the limit of the NtHMA mutant plant, the growth characteristics of plants with the mutated NtHMAα and/or NtHMAβ gene(s), and plants with both the mutated NtPCSs and NtPCSt genes were compared to those of control groups. In detail, the plant height, the number of leaves, and the weight of leaves of five individuals for each plant type were measured and compared.

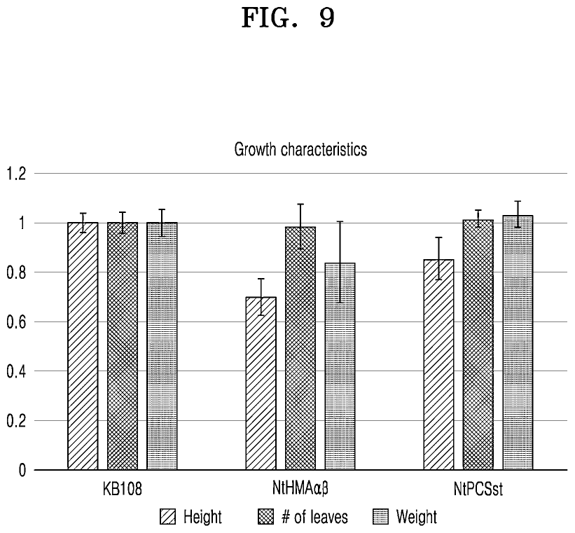

As a result, as shown in FIG. 9 and Table 13 below, the plants with both the mutated NtHMAα and NtHMAβ genes showed a 30% decrease in the plant height, no significant difference in the number of leaves, and a 16% decrease in the weight of the leaves, as compared with the control group. These results indicate that plants with the mutated NtHMAα and NtHMAβ genes exhibit growth inhibition. In contrast, the plants with both the mutated NtPCSs and NtPCSt genes showed a 15% slight decrease in the plant height, but no significant changes in the number and weight of leaves, as compared with the control group, indicating no growth inhibition.

TABLE 13

| | | Height | Number of | Weight of |
|---|---|---|---|---|
| No. | Plant | (cm) | leaves | leaves (g) |
| 1 | KB108 | 62 | 12 | 145.5 |
| 2 | (Control) | 66 | 13 | 149.0 |
| 3 | | 63 | 13 | 142.0 |
| 4 | | 68 | 13 | 142.0 |
| 5 | | 67 | 12 | 129.0 |
| Mean | | 65.2 | 12.6 | 141.5 |
| 1 | NtHMAα | 45 | 13 | 146.0 |
| 2 | Mutant | 55 | 13 | 167.0 |
| 3 | | 53 | 13 | 154.0 |
| 4 | | 52 | 13 | 156.0 |
| 5 | | 53 | 13 | 163.0 |
| Mean | | 51.6 | 13 | 157.2 |
| 1 | NtHMAβ | 58 | 12 | 144.6 |
| 2 | Mutant | 51 | 14 | 169.0 |
| 3 | | 57 | 13 | 176.0 |
| 4 | | 58 | 14 | 172.0 |
| 5 | | 54 | 13 | 160.0 |
| Mean | | 55.6 | 13.2 | 164.32 |
| 1 | NtHMAαβ | 50 | 11 | 108.5 |
| 2 | Mutant | 50 | 12 | 157.5 |
| 3 | | 40 | 14 | 123.0 |
| 4 | | 41 | 12 | 107.0 |
| 5 | | 47 | 13 | 99.0 |
| Mean | | 45.6 | 12.4 | 117.0 |
| 1 | NtPCSst | 61 | 13 | 145.0 |
| 2 | Mutant | 60 | 13 | 158.0 |
| 3 | | 57 | 13 | 149.0 |
| 4 | | 54 | 13 | 142.0 |
| 5 | | 47 | 12 | 138.0 |

| | | | |
|---|---|---|---|
| Mean | 55.8 | 12.8 | 146.4 |

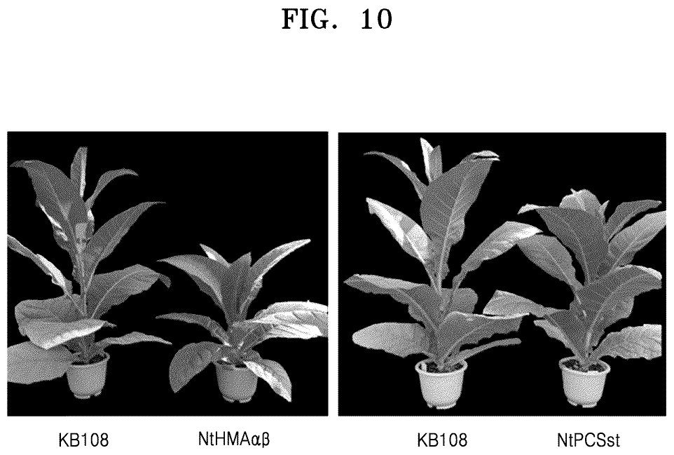

In addition, as shown in FIG. 10, the NtHMAαβ variant showed growth inhibition detectable with the naked eye, as compared with the control group, whereas the NtPCSst variant showed no significant difference in the growth, as compared with the control group.

1. A plant cell in which expression or activity of a phytochelatin synthase (PCS) gene or a protein encoded by the PCS gene is reduced, as compared with a parent cell.

2. The plant cell of claim 1, wherein the PCS gene is a PCS gene derived from Nicotiana sylvestris (NtPCSs), a PCS gene derived from Nicotiana tomentosiformis (NtPCSt), or a combination thereof (NtPCSst).

3. The plant cell of claim 1, wherein the plant cell is genetically engineered by at least one selected from the group consisting of an RNA interference (RNAi) system, a meganuclease system, a zinc finger nuclease system, a transcription activator-like effector nuclease (TALEN) system, a CRISPR/Cas9 system, X-ray irradiation, gamma-ray irradiation, ethyl methanesulfonate treatment, and dimethyl sulfate treatment.

4. The plant cell of claim 1, wherein the plant is Nicotiana tabacum.

5. A plant having reduced heavy metal absorption, comprising the plant cell of claim 1.

6. A method of reducing heavy metals in a plant, the method comprising reducing expression or activity of a PCS gene or a protein encoded by the PCS gene, as compared with a parent cell of the plant.

7. The method of claim 6, wherein the reducing of expression or activity of the PCS gene or the protein encoded by the PCS gene is performed by at least one selected from the group consisting of an RNA interference (RNAi) system, a meganuclease system, a zinc finger nuclease system, a transcription activator-like effector nuclease (TALEN) system, a CRISPR/Cas9 system, X-ray irradiation, gamma-ray irradiation, ethyl methanesulfonate treatment, and dimethyl sulfate treatment.

8. The method of claim 6, wherein the heavy metal is one or more selected from the group consisting of cadmium, arsenic, antimony, lead, mercury, chromium, tin, zinc, barium, bismuth, nickel, cobalt, manganese, iron, copper, and vanadium.

9. A CRISPR-Cas9 recombinant vector comprising a single guide RNA targeting a PCS gene.

10. The CRISPR-Cas9 recombinant vector of claim 9, wherein the PCS gene is a PCS gene derived from Nicotiana sylvestris (NtPCSs) or a PCS gene derived from Nicotiana tomentosiformis (NtPCSt).

11. The CRISPR-Cas9 recombinant vector of claim 10, wherein the sgRNA targeting the NtPCSs gene consists of a nucleotide sequence of SEQ ID NO: 1 or SEQ ID NO: 2, the sgRNA targeting the NtPCSt gene consists of a nucleotide sequence of SEQ ID NO: 3 or SEQ ID NO: 4, and the sgRNA targeting the NtPCSs gene and the NtPCSt gene consists of a nucleotide sequence of SEQ ID NO: 5 or SEQ ID NO: 6.

12. A method of preparing a plant having reduced heavy metal absorption, the method comprising transforming a plant cell with the recombinant vector of claim 9.
